(19) United States
(12) Patent Application Publication
(10) Pub. No.: US 2008/0249165 A1
(43) Pub. Date: Oct. 9, 2008

(54) Glycosides and Salts Thereof

(75) Inventors: Janos Kuszmann, Budapest (HU); Istvan Kurucz, Budapest (HU); Gabor Medgyes, Budapest (HU); Nicholas Bodor, Bal Harbour, FL (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Ivax Drug Research Institute Ltd., Budapest (HU)

(21) Appl. No.: 11/659,453

(22) PCT Filed: Aug. 5, 2005
(86) PCT No.: PCT/US05/27879

§ 371 Date: Mar. 14, 2008

Related U.S. Application Data
(60) Provisional application No. 60/599,147, filed on Aug. 5, 2004.

(51) Int. Cl. A61K 31/351; C07D 407/12; A61P 11/00; C07D 407/14

(52) U.S. Cl. 514/460; 549/414

(57) ABSTRACT

The invention relates to new polysulfated glycosides of formula (I), the salts thereof formed with alkali metals or alkaline-earth metals, as well as the pharmaceutical compositions containing these compounds as active ingredients. Furthermore the invention provides a method of preventing, treating or alleviating the symptoms of acute and chronic inflammatory disorders of the airways of mammals—including asthma and asthma-related pathologies.

### BACKGROUND OF THE INVENTION

1. Field of Invention

The present invention relates to glycosides, the salts thereof, and the pharmaceutical compositions containing these glycosides as active ingredients. Furthermore the invention provides a method of preventing, treating or alleviating the symptoms of acute and chronic inflammatory disorders of the airways of mammals—including asthma and asthma-related pathologies.

2. Summary of Related Art

Inflammation is a multi-step cascade process, any part of which may be the subject of potential therapeutic intervention. Briefly, inflammation entails the infiltration of immunologically competent cells (for example eosinophils, mast cells, activated T-lymphocytes) into the injury site where they, together with resident cells, release bioactive mediator substances (e.g., histamine, proteases, a host of cytokines and chemokines), which increase the permeability of nearby blood vessels, attract and stimulate bystander cells. The altered permeability of vessels results in a fluid exudate forming at the injury site followed by a further influx of reactive leukocytes and their eventual efflux into the damaged area. (For an overview see, Trowbridge and Emling, Inflammation: A Review of the Process Quintessence Pub, Co., 1997). Secretion of collagen and mucus by, and proliferation of, resident cells (smooth muscle and epithelial cells or fibroblasts stimulated by the released mediators) establish the extension of pathological alterations (e.g., airway obstruction) and contribute to their development.

Inflammation is associated with a variety of pulmonary conditions including e.g., intrinsic or extrinsic asthma bronchiale, any inflammatory lung disease, acute or chronic bronchitis, pulmonary inflammatory reactions secondary to chronic bronchitis, chronic obstructive lung disease, pulmonary fibrosis, as well as any pulmonary condition in which white blood cells may play a role including, but not limited to, idiopathic pulmonary fibrosis and any other autoimmune lung disease. Asthma is one of the most common forms of pulmonary inflammation affecting the large and small airways of the lung. It impacts on 5% to 10% of the human population, resulting in an estimated 27 million patient visits, 6 million lost workdays, and 90.5 million days of restricted activity per year. The morbidity and mortality rates for asthma are growing worldwide (Plaut and Zimmerman, “Allergy and Mechanisms of Hypersensitivity” in Fundamental Immunology, 3^{rd} Ed., Paul (ed.), Raven Press, New York, N.Y., at 1399 (1993)).

Conventional anti-asthma treatments have been predicated on the strict avoidance of all triggering allergens, which is inherently difficult to achieve, and on therapeutic regimens based on pharmacological agents having unfortunate side effects and suboptimal pharmacokinetic properties. β₂-adrenergic agonists used to treat bronchospasm have no effect on airway inflammation or bronchial hyperreactivity (Palmer et al., New Engl. J. Med. 331:1314 (1994)). Also, regular or prolonged use of β₂-adrenergic agonists is associated with poor control of asthma, increase in airway hyperresponsiveness to allergen, and reduced bronchoconstriction protection (Bhagat et al., Chest 108:1235 (1995)). Moreover, chronic use of β₂-adrenergic agents alone, by causing down regulation of β₂-adrenergic receptors, is suspected to worsen bronchial hyperreactivity. Theophylline (an anti-asthma methylxanthine) is characterized by substantial variability in its absorbance and clearance. Corticosteroids, while relatively safe in adult patients, are toxic for children, resulting in adrenal suppression and reduced bone density and growth (Woolock et al., am. Respir. Crit. Care Med. 153:1481 (1996)). Cromolyn, used to prevent asthmatic episodes, is effective in preventing an asthmatic reaction only if given prior to an attack (Volcheck et al., Postgrad Med. 104(3):127 (1998)). Antihistamines occasionally prevent or abort allergic asthmatic episodes, particularly in children, but often are only partially effective because histamines are only one of many inflammation associated mediators (Cuss, “The Pharmacology of Antiasthma Medications”, in Asthma as an Inflammatory Disease, O'Byrne, Ed., Dekker, Inc., New York, at 199 (1990)) and O'Byrne, “Airway Inflammation and Asthma”, in Asthma as an Inflammatory Disease, O'Byrne, Ed., Dekker, Inc., New York, N.Y., 143 (1990)).

Thus, current drug modalities suffer from a number of drawbacks. In general, conventional agents have a relatively short duration of action and may be partially or wholly ineffective when administered after antigen challenge occurs. Moreover, because of serious adverse effects associated with the use of agents such as β₂-adrenergic agonists and corticosteroids, therapeutic margins of safety with such agents are relatively narrow and patients using such agents must be carefully monitored (see e.g., WO 94/06783, WO 99/06025, U.S. Pat. Nos. 5,690,910 and 5,980,865). In a recent clinical study, with inhaled corticosteroids, only transient improvement occurred in the airways function of 5-11-year-old asthmatic children after the first year of therapy, with regression to that observed with placebo over the next 3 years (The Childhood Asthma Management Program Research Group, N. Engl. J. Med., 343:1054 (2000)). This regression can best be explained by remodeling changes (characteristic feature of asthma) occurring in the airways that are refractory to corticosteroids (Davies, Curr. Opin. Allergy Clin. Immunol, 1:67 (2001)).

It is known from relevant literature, that certain mixtures of polysulfated disaccharides which were synthesized by nitrous acid treatment of such natural products as for example heparin or heparin sulfate, followed by reduction with borohydride and subsequent sulfation of the partially purified samples (U.S. Pat. No. 5,690,910; U.S. Pat. No. 5,980,865 and WO 02/083700)—displayed antiinflammatory effect in different asthma models. WO 02/08370 discloses and claims uronic acid derivatives which require a carboxylic acid moiety or salt thereof on the molecules and relates to sulfate and phosphate esters thereof.

### SUMMARY OF THE INVENTION

The present invention relates to novel glycosides processes to make such compounds, and pharmaceutical compositions containing such compounds, which have more favourable pharmacological properties and less undesirable side-effects, than known anti-asthmatics. The invention further relates to methods of treating patients in need of treatment comprising administering the novel compounds and compositions of the invention to said patients.

### DETAILED DESCRIPTION OF THE INVENTION

The invention relates to novel glycosides of formula (I),

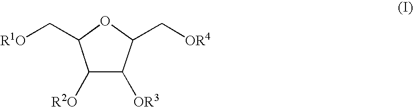

wherein R¹, R², R³ and R⁴, independently of each other, stand for H, C₁₋₄ alkyl[?], —SO₃H, sulfated or unsulfated glycosyl or sulfated or unsulfated diglycosyl group—with the proviso, that at least one of R¹-R⁴ is a sulfated or unsulfated glycosyl or sulfated or unsulfated diglycosyl group—as well as the isomers and pharmaceutically acceptable salts thereof. The term “pharmaceutically acceptable salts” includes, for example, alkali salts and alkaline earth metal salts as well as any other pharmaceutically acceptable counterion or counterions associated with one or more of the sulfate groups on the molecule.

As all of the four carbon atoms of the tetrahydrofuran ring represent chiral centers, obviously all possible stereoisomers (allit, dulcit, idit, mannit, sorbit and talit as well as D- and L-enantiomers thereof are covered by the formula (I). The term “isomer” herein includes all such compounds and variants thereof in the compound of formula (I).

The meaning of sulfated glycosyl group can be any pentopyranose or hexopyranose molecule with optional configuration, in which one or more of the hydroxyl groups are present as an O-sulfate ester and the sugar moiety is attached to the aglycon with its anomeric carbon atom via an α- or β-linkage. The unsulfated glycosyl group contains all hydroxyl groups or protected versions thereof. The unsulfated compounds are useful as intermediates to produce the sulfated compounds recited herein.

The meaning of sulfated diglycosyl group can be any pentopyranose or hexopyranose molecule with optional configuration, one of the hydroxyl group of which is glycosylated with a further pentopyranose or hexopyranose molecule with optional configuration, and one or more of the hydroxyl groups of the so formed diglycosyl unit are present as an O-sulfate ester and the sugar moiety is attached to the aglycon with its anomeric carbon via α- or β-linkage. The unsulfated diglycosyl group contains all hydroxyl groups or protected versions thereof. The unsulfated compounds are useful as intermediates to produce the sulfated compounds of the invention. The compounds of formula (VII)-(XIX) with R¹, R² and R³ equal to H provide examples of these useful intermediates with an unsulfated glycosyl or diglycosyl moiety linked to the aglycon via the anomeric carbon atom.

All possible stereoisomers (arabino-, lyxo-, ribo- and xylo-) are included in the structure of pentoses, as well as D- and L-enantiomers thereof. Similarly all possible stereoisomers (allo-, altro-, galacto-, gluco-, gulo-, ido-, manno- and tallo-) are included in the structure of hexoses, as well as D- and L-enantiomers thereof. The term “isomer” includes all such compounds and variants thereof in the compound of formula (I).

Alkali metal salts of the compounds of the Invention mean Na, K or Li salts, while alkaline-earth metal salts preferably are Mg and Ca salts.

Those compounds of formula (I), as well as alkali metal and alkaline-earth metal salts thereof, wherein R¹, R³ and R⁴ stand for —SO₃H and R² is a polysulfated glycosyl group, represent a preferred group of the compounds of the invention.

Those compounds of formula (I), as well as alkali metal and alkaline-earth metal salts thereof, wherein R¹, R³ and R⁴ stand for —SO₃H and meaning R² is a polysulfated diglycosyl group, represent a further preferred group of the compounds of the invention.

Those compounds of formula (I), as well as alkali metal and alkaline-earth metal salts thereof, wherein R¹ and R⁴ represent polysulfated glycosyl or diglycosyl group and R² and R³ represent —SO₃H, are a further preferred group of the compounds of the invention.

Those compounds of formula (I), as well as alkali metal and alkaline-earth metal salts thereof, wherein R¹, R² and R⁴ stand for polysulfated glycosyl group, while the meaning of R³ is —SO₃H, represent a further preferred group of the compounds of the invention.

Especially preferred representatives of the compounds of formula (I) of the present invention are—without limitation—the following:
2,5-Anhydro-1,4,6-tri-O-sulfato-3-O-(2,3,4,6-tetra-O-sulfato-α-L-idopyranosyl)-D-mannitol hepta sodium salt
2,5-Anhydro-1,4,6-tri-O-sulfato-3-O-(2,3,4,6-tetra-O-sulfato-α-L-idopyranosyl)-D-mannitol hepta potassium salt
2,5-Anhydro-1,4,6-tri-O-sulfato-3-O-(2,3,4,6-tetra-O-sulfato-β-D-glucopyranosyl)-D-mannitol hepta sodium salt
2,5-Anhydro-1,4,6-tri-O-sulfato-3-O-(2,3,4-tri-O-sulfato-α-L-arabinopyranosyl)-D-mannitol hexa potassium salt
2,5-Anhydro-1,4,6-tri-O-sulfato-3-O-(2,3,4-tri-O-sulfato-α-D-arabinopyranosyl)-D-mannitol hexa potassium salt
2,5-Anhydro-3,4,6-tri-O-sulfato-1-O-(2′,3′,4′,6′,2,3,6-hepta-O-sulfato-β-D-maltosyl)-D-mannitol deca potassium salt
2,5-Anhydro-1,4,6-tri-O-sulfato-3-O-(2′, 3′,4′,6′,2,3,6-hepta-O-sulfato-β-D-maltosyl)-D-mannitol deca potassium salt
2,5-Anhydro-1,4,6-tri-O-sulfato-3-O-[6-O-(2,3,4,6-tetra-O-sulfato-β-D-glucopyranosido)-2,3,4-tetra-O-sulfato-β-D-glucopyranosyl]-D-mannitol deca potassium salt
2,5-Anhydro-3,4-di-O-sulfato-1,6-bis-O-(2,3,4,6-tetra-O-sulfato-β-D-glucopyranosyl)-D-mannitol deca potassium salt
2,5-Anhydro-3,4-di-O-sulfato-1,6-bis-O-(2,3,4,6-tetra-O-sulfato-α-L-idopyranosyl)-D-mannitol deca potassium salt
2,5-Anhydro-3,4-di-O-sulfato-1,6-bis-[(6-O-2,3,4,6-tetra-O-sulfato-β-D-glucopyranosido)-2,3,4-tri-O-sulfato-β-D-glucopyranosyl]-D-mannitol hexadeca potassium salt
2,5-Anhydro-3,4-di-O-sulfato-1,6-bis-O-[2′,3′,4′,6′,2,3,4-hepta-O-sulfato-β-D-maltosyl]-D-mannitol hexadeca potassium salt
2,5-Anhydro-4-O-sulfato-1,3,6-tri-O-[2,3,4,6-tetra-O-sulfato-β-D-glucopyranosyl]-D-mannitol trideca potassium salt

Compounds of formula (I) of the present invention can be synthesized from compounds of formula (VI)

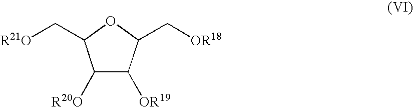

—wherein R¹⁸, R¹⁹, R²⁰ and R²¹, independently of each other, stand for hydrogen atom, glycosyl or diglycosyl group, and at least one of R¹⁸-R²¹ is other, than hydrogen atom—by transforming its free hydroxyl groups into sulfate esters using known methods.

Sulfur trioxide or an adduct thereof formed with an organic base (for example triethylamine or pyridine) or with dimethylformamide can be used as reagent for the preparation of O-sulfate esters.

In given case monofunctional acidic esters obtained by the above methods can be transformed into salts for example with alkali metal or alkali earth-metal acetates. After purification, the salts can be obtained by freeze drying, precipitation or crystallization.

Compounds of formula (VI), used as starting materials in the above process for the synthesis of compounds of formula (I) of the present invention, are also new. They can be synthesized for example by the following, known methods:

a) Those compounds of formula (VI), wherein R¹⁸, R¹⁹, R²¹ stand for hydrogen atom and R²⁰ represents glycosyl group, can be synthesized for example by using a compound of formula (II) or (III)

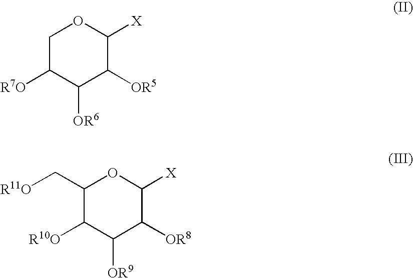

—wherein X can be halogen atom, trichloroacetimidate or phenylthio group and R⁵-R¹¹ represent aliphatic or aromatic ester or ether group—as donor molecule and a compound of formula (IV)

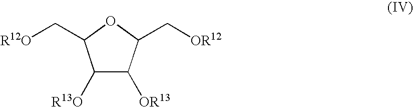

—wherein R¹² represents —C(O)R wherein R is C₁-C₄ alkyl or C₆-C₁₂ alkyl aryl or R¹² represents C₁-C₆ alkyl or C₆-C₁₂ alkyl aryl protecting group, while R¹³ represents hydrogen atom—as acceptor, and the glycosylation is carried out in the presence of appropriate activators. Then the protective groups are cleaved from the so obtained compound of formula (V)

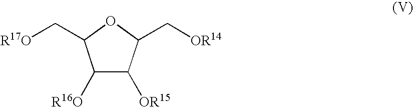

—wherein R¹⁴ and R¹⁷ represents —C(O)R wherein R is C₁-C₄ alkyl or C₆-C₁₂ alkyl aryl, or R¹⁴ and R¹⁷ represents C₁-C₆ alkyl or C₆-C₁₂ alkyl aryl and one of R¹⁵ and R¹⁶ represents protected glycosyl group and the other represents a hydrogen atom.
b) Those compounds of formula (VI), wherein R¹⁸, R¹⁹, R²¹ represent hydrogen atoms and R²⁰ represents diglycosyl group, can be synthesized for example by carrying out the glycosylation according to process a), but using a compound of formula (III)—wherein one of R⁸, R⁹, R¹⁰ and R¹¹ represents a protected hexopyranosyl group, while the others represent ester groups—as donor molecule.
c) Those compounds of formula (VI), wherein R¹⁹ and R²⁰ stand for hydrogen atom and R¹⁸ and R²¹ represent glycosyl group, can be synthesized for example by using a compound of formula (II) or (III)—wherein the meaning of X and R⁵-R¹¹ as described above—as donor molecule and a compound of formula (IV)—wherein R¹³ represents —C(O)R wherein R is C₁-C₄ alkyl or C₆-C₁₂ alkyl aryl, or R¹³ represents C₁-C₆ alkyl or C₆-C₁₂ alkyl aryl while R¹² represents hydrogen atom—as acceptor and the glycosylation is carried out in the presence of appropriate activators. Then the protective groups are cleaved from the so obtained compound of formula (V)—wherein R¹⁴ and R¹⁷ represent protected glycosyl groups and R¹⁵ and R¹⁶ represents —C(O)R wherein R is C₁-C₄ alkyl or C₆-C₁₂ alkyl aryl, or R¹⁵ and R¹⁶ represents C₁-C₆ alkyl or C₆-C₁₂ alkyl aryl.
d) Those compounds of formula (VI), wherein R¹⁹ represents a hydrogen atom and R¹⁸, R²⁰ and R²¹ represent glycosyl groups, can be synthesized for example by using a compound of formula (II) or (III)—wherein the meaning of X and R⁵-R¹¹ as described above—as donor molecule and a compound of formula (IV)—wherein R¹² and R¹³ represent hydrogen atoms—as acceptor and the glycosylation is carried out in the presence of appropriate activators. Then the protective groups are cleaved from the so obtained compound of formula (V)—wherein R¹⁴, R¹⁶ and R¹⁷ represent protected glycosyl groups and R¹⁵ represents hydrogen atom.

In the above glycosylation reactions mercury or silver salts, boron trifluoride diethyl etherate, N-iodosuccinimide and trifluoromethanesulfonic acid or the mixture of the latter two can be used as activator.

The cleavage of the protective groups can be carried out by acid hydrolysis or reduction in the presence of a catalyst in the case of ethers and acetals, while in the case of esters Zemplén's method (base catalysed trans-esterification) or hydrolysis in the presence of a base can be used.

Abbreviations used in the description:

Ac=acetyl
Bz=benzoyl
Bn=benzyl
Me=methyl
Ph=phenyl

### NIS=N-iodosuccinimide

TfOH=trifluoromethanesulfonic acid

As used in this specification, the singular forms “a”, “an” and “the” specifically also encompass the plural forms of the terms to which they refer, unless the content clearly dictates otherwise. For example, reference to “a modulator” includes mixtures of modulators.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, unless specifically indicated otherwise, the word “or” is used in the “inclusive” sense of “and/or” and not the “exclusive” sense of “either/or.”

As used herein, the terms “treating” or “treatment” are used to indicate reducing, alleviating, preventing, inhibiting the development of and/or reversing the symptoms of a condition. Conditions to be treated by the methods and compositions of the invention include any condition characterized by, or including, acute and chronic inflammatory disorders of the airways. Hence, the terms “inflammatory disorder” or “inflammatory disorders of the airways” encompass any inflammatory lung disease, including asthma, intrinsic or extrinsic asthma bronchiale, acute chronic bronchitis, allergic rhinitis, pulmonary inflammatory and structural reactions secondary to chronic bronchitis, chronic obstructive lung disease, pulmonary fibrosis. The present invention is also useful for any pulmonary condition in which white blood cells and airway remodeling may play a role including but not limited to idiopathic pulmonary fibrosis and any other autoimmune lung disease.

By “asthma” is meant a condition of allergic origins, the symptoms of which include continuous or paroxysmal labored breathing accompanied by wheezing, a sense of constriction in the chest, and often attacks of coughing or gasping. By “asthma-related pathology” is meant a condition whose symptoms are predominantly inflammatory in nature with associated bronchospasm. Hence, both asthma and asthma-related pathologies are characterized by symptoms that include narrowing of airways, due in varying degrees to contraction (spasm) of smooth muscle, edema of the mucosa, including that of the upper airways and mucus in the lumen of the bronchi and bronchioles. Non-limiting representative examples of “asthma-related pathologies” include non-asthmatic conditions characterized by airway hyperresponsiveness (e.g., chronic bronchitis, emphysema, cystic fibrosis and respiratory distress).

Compositions and methods taught herein are exemplified, for asthma. However, the invention should not be construed as limited to this particular pulmonary disease. Asthma offers the advantage of having been studied extensively and provides several accepted models to evaluate the invention. It is known that sensitization and allergen challenge leads to airway hyperresponsiveness to various agonists. Hence, acetylcholine, known as a spasmogenic agent, is capable of inducing larger contractions of the muscle cells in tissues obtained from the trachea of sacrificed animals (which had been sensitized to provoke airway hyper-responsiveness) than from control animals following allergen challenge (see, e.g. Tokuoka et al., Br. J. Pharmacol. 134:1580 (2001); Nakata et al., Int. Immunol. 13:329 (2001); Emala and Hirshman, Monogr. Allergy 33:35 (1996)).

The most prominent characteristic of asthma is bronchospasm, or narrowing of the airways. Asthmatic patients have prominent contraction of the smooth muscles of large and small airways, increased mucus production, and increased inflammation (Plaut and Zimmerman, supra). The inflammatory response in asthma is typical for tissues covered by a mucosa and is characterized by vasodilation, plasma exudation, recruitment of inflammatory cells such as neutrophils, monocytes, macrophages, lymphocytes, and eosinophils to the sites of inflammation, and the release of inflammatory mediators by resident tissue cells (e.g., mast cells or airways epithelial cells) or by migrating inflammatory cells (Hogg, “Pathology of Asthma”, in Asthma as an Inflammatory Disease, O'Byrne (ed.), Marcel Dekker, Inc., New York, N.Y., at 1 (1990)). Asthma may be triggered by a variety of causes such as allergic reactions, a secondary response to infections, industrial or occupational exposures, ingestion of certain chemicals or drugs, exercise (Hargreave et al., J. Allergy Clin. Immunol. 83:1013 (1986)).

The compounds of formula (I) according to the invention have also been found effective to decrease mucus production of bronchial epithelial cells and to inhibit growth factor mediated proliferation of smooth muscle cells.

An increase in bronchial hyperreactivity (AHR), the hallmark of a more severe form of asthma, can be induced by both airway antigenic and non-antigenic stimuli. Late phase response and persistent hyperresponsiveness in allergen-induced asthma have been associated with the recruitment of leukocytes, and particularly eosinophils, to inflamed lung tissue (Abraham et al., Am. Rev. Respir. Dis. 138:1565 (1988)). Eosinophils release several inflammatory mediators including 15-HETE, leukotriene C4, PAF, cationic proteins, eosinophil peroxidase.

The terms “antigen” and “allergen” are used interchangeably to describe those molecules, such as dust or pollen that can induce an allergic reaction and/or induce asthmatic symptoms in an individual suffering from asthma. Thus, an asthmatic individual “challenged” with an allergen or an antigen is exposed to a sufficient amount of the allergen or antigen to induce an asthmatic response. The compounds of formula (I) according to the invention have been found effective to treat AHR subsequent to ovalbumin sensitization and antigen challenge.

The biological activity of the compounds of formula (I) of the present invention in different animal models is demonstrated below on the compound of Example 1.

### Model 1

### Examination of the Effect of Locally Administered Polysulfated Glycosides on Airways' Hyper-Responsiveness Ex Vivo

Inflammation of the airways may lead to bronchial hyper-responsiveness, which is a characteristic feature of asthma.

Brown Norway (BN) rats were actively sensitized to ovalbumin (OA) by a subcutaneous injection of 0.5 ml of OA/Al(OH)₃ gel mixture (2 mg OA+10 g Al(OH)₃/100 ml saline) on day 1 with subsequent subcutaneous injections (10 mg OA+10 g Al(OH)₃/100 ml saline) given on days 14 and 21. On day 28, animals received the compound described in the first example intratracheally (0.001; 0.01; 0.1 or 1.0 mg/kg dose) 2 hours before antigen challenge. Antigen challenge was performed by inhalation of nebulised ovalbumin (1% antigen solution administered in a TSE inhalation system for 1 hour). Animals were sacrificed 48 hours post antigen challenge wherein the tracheas were removed to an organ bath. Dissected tracheas were allowed to equilibrate for 30 minutes before measuring tracheal spasmogenic response curves to acetylcholine (Ach).

As shown in Table 1 ovalbumin challenge of sensitized animals in this model caused a significant tracheal hyper-reactivity to acetylcholine, when the response to the spasmogenic agent was determined 48 h after antigen challenge. The compound described in the first example in a dose of 0.1 mg/kg, brought this elevation back to control level.

TABLE 1
Effect of antigen challenge and intratracheal pretreatment
with compound of Example 1 on the tracheal contraction
to acetylcholine in BN-rats

| | | | Examined compound | |
|---|---|---|---|---|
| Parameters | Control | Placebo | 0.001 mg/kg | 0.1 mg/kg |
| ED₅₀* | 5.18 ± 0.12 | 5.80 ± 0.36 | 5.78 ± 0.30 | 4.70 ± 0.37 |
| p | 0.032 | | NS*** | 0.027 |
| MAX** | 100 ± 0 | 192 ± 34 | 166 ± 41 | 99 ± 16 |
| p | 0.001 | | NS*** | 0.014 |

*log M acetylcholine (Ach), causing 50% contraction relative to control (mean ± SEM)
**Contraction at maximal Ach concentration relative to control (mean ± SEM)
***Non-significant

### Model 2.

### Examination of the Effect of Polysulfated Glycosides on the Allergen Stimulated Mucus Production of Airways Epithelial Cells.

In a sensitized animal antigenic challenge results in mucus production of airways epithelial cells, which is a characteristic feature of allergic asthma.

Sensitized BN rats were treated intratracheally with varying (0.001-1.0 mg/kg) dose of compound described in the first example, two hours before antigenic challenge, using a similar protocol described in Model 1. Lungs were collected 48 hours after challenge and were fixed in 8% phosphate buffered formaldehyde. Samples were then processed for histochemistry routinely. 5 μm thick sections were stained with periodic-acid-Schiff (PAS) reagents and were counterstained with haematoxylin-eosine. On the sections each epithelial cells of the airways were counted in the whole preparation at a magnification of 400×. The number of PAS(+) [mucus producing] epithelial cells was expressed as the ratio of the total number of epithelial cells.

As it is shown in Table 2, allergen challenge stimulates the mucus production of airways epithelial cells (control vs. challenge). At the dose of 0.1 mg/kg the compound significantly decreased the number of PAS(+), mucus producing cells.

TABLE 2
Effect of antigen challenge and intratracheal treatment
with compound of Example 1, on the allergen induced mucus
production of airways epithelial cells in BN rats

| Groups | Dose mg/kg | %* | p-value |
|---|---|---|---|
| Control | | 2.9 ± 0.7 | <0.001 |
| Challenge | | 32.1 ± 3.0 | — |
| Treated | 0.001 mg/kg | 35.2 ± 3.5 | NS |
| | 0.1 mg/kg | 17.5 ± 2.4 | <0.001 |

*number of PAS(+) cells as percent of total number of cells (average ± SEM)

### Model 3.

### Examination of the Effect of Polysulfated Glycosides on the Extent of Perivascular Oedema Developed in Asthmatic Lung Tissue.

In a sensitized animal antigen challenge, as a result of the developing inflammatory processes, increases the permeability of the blood vessels resulting in plasma excudation around the periphery of the vasculature.

Sensitized BN rats were treated intratracheally with varying (0.001-1.0 mg/kg) dose of compound described in the first example, two hours before antigenic challenge, using a similar protocol described in Model 1. Lungs were collected 48 hours after challenge and were fixed in 8% phosphate buffered formaldehyde. Samples were then processed for histochemistry routinely. 5 μm thick sections were stained with periodic-acid-Schiff (PAS) reagents and were counterstained with haematoxylin-eosine. On the sections the area of the connective tissue around the vasculare was determined and expressed as a ratio of the area of the corresponding blood vessel itself.

As it is shown in Table 3, allergen challenge causes aedema around the vasculature, the extent of which was significantly decreased even at the smallest dose of the examined compound.

TABLE 3
Effect of antigenic challenge and intratracheal
treatment with compound of Example 1, on the
extent of developing oedema in BN rats

| Groups | Dose mg/kg | Oedema* | p-value |
|---|---|---|---|
| Control | | 41 ± 6 | <0.001 |
| Challenge | | 156 ± 9 | — |
| Treated | 0.001 mg/kg | 41 ± 6 | <0.001 |
| | 0.1 mg/kg | 53 ± 6 | <0.001 |

*area of oedema relative to area of vasculature (average ± SEM)

### Model 4.

### Examination of the Effect of Polysulfated Glycosides on the Extent of Peribronchial Eosinophilia Developed in Asthmatic Lung Tissue.

In a sensitized animal antigenic challenge results in the infiltration of eosinophils into the lung and this phenomenon is one of the most typical feature of asthma.

Sensitized BN rats were treated intratracheally with varying (0.001-1.0 mg/kg) dose of compound described in the first example, two hours before antigenic challenge, using a similar protocol described in Model 1. Lungs were collected 48 hours after challenge and were fixed in 8% phosphate buffered formaldehyde. Samples were then processed for histochemistry routinely. 5 μm thick sections were stained with May Gruenvald Giemsa and the number of eosinophils, situated peribronchially, was determined.

As it is shown in Table 4, allergen challenge causes an extraordinary increase in the number of peribronchially situated eosinophils in the lung. Treatment with compound of Example 1, already at the smallest dose decreases the extent of it, at higher doses the decrease become statistically significant.

TABLE 4
Effect of antigen challenge and intratracheal treatment
with compound of Example 1, on the extent of the developing
peribronchial eosinophilia in BN rats

| Groups | Dose mg/kg | Oedema* | p-value |
|---|---|---|---|
| Control | | 2 ± 0 | <0.001 |
| Challenge | | 81 ± 3 | — |
| Treated | 0.001 mg/kg | 74 ± 2 | NS |
| | 0.1 mg/kg | 25 ± 2 | <0.001 |

*eosinophil number/visual field (average ± SEM)

### Model 5.

### IP-3 Receptor Antagonistic Effect of Polysulfated Glycosides

The polysulfated glycosides of the present invention, depending on their chemical structure, inhibit the binding of inositol-1,4,5-trisphosphate (IP3) to its receptor in microsomal membrane preparations. As IP3 is a messenger molecule playing distinguished role in the activation of different cells, interfering with this function can explain the anti-asthmatic effect of these polysulfated glycosides.

The IP3 antagonist effect of the polysulfated glycosides was determined using rat cerebellum membrane preparations according to Worley et al. (JBC 262, 12132, 1987). As is seen in Table 5, all the compounds described in Examples 1-10 possess varying IP3 antagonist activity.

TABLE 5
IP-3 receptor antagonistic effect of polysulfated glycosides

| Compound | IC₅₀ (μg/ml) | Average IC₅₀ |
|---|---|---|
| (Number of example) | Average ± SEM (n) | (nM) |
| 1 | 1.55 ± 0.27 (4) | 1489 |
| 2 | 5.01 ± 1.32 (4) | 4813 |
| 3 | 1.26 ± 0.20 (3) | 1388 |
| 4 | 20.43 ± 2.73 (3) | 20328 |
| 5 | 26.66 ± 5.24 (4) | 26527 |
| 6 | 0.37 ± 0.13 (5) | 222 |
| 7 | 0.40 ± 0.10 (3) | 240 |
| 8 | 0.90 ± 0.26 (6) | 539 |
| 9 | 0.31 ± 0.00 (3) | 115 |
| 10 | 0.30 ± 0.09 (4) | 137 |

The compounds according to the invention are optimally formulated in a pharmaceutically acceptable vehicle with any type of well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating compositions of the invention will vary depending upon the mode of administration of the composition to a mammal, generally pharmaceutically acceptable carriers are physiologically inert and non-toxic. Formulations of compositions according to the invention may contain more than one type of compound of the invention, as well as any other pharmacologically active ingredient useful for the treatment of the particular pulmonary inflammation being treated. Such compounds may include without limitation, β-adrenoceptor antagonists: albuterol, metaproterenol, levalbuterol, pirbuterol, salmeterol, bitolterol; glucocorticoids: beclomethasone, triamcinolone, flunisolide, budesonide, fluticasone; leukotriene-receptor antagonists and leukotriene-synthesis inhibitors: zafirlukast, montelukast, zileutin; other anti-asthmatics: cromolyn, nedocromil, theophylline; anti-cholinergic agents: ipratropium, oxitropium, tiotropium; H₁ receptor antagonist anti-histamines: diphenhydramine, pyrilamine, promethazine, loratidine, chlorocyclizine, chloropheniramine, fexofenadine and adrenocorticosteroids.

The compositions of the invention can be administered by standard routes (e.g. oral, inhalation, rectal, nasal, topical, including buccal and sublingual, or parenteral, including subcutaneous, intramuscular, intravenous, intradermal, transdermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powder and aerosols. The present invention encompasses liquid and powdered compositions suitable for nebulization and intrabronchial use, or aerosol compositions administered via an aerosol unit dispensing metered doses (“MDI”). Particularly preferred devices contemplated are described in U.S. Pat. No. 5,447,150.

The active ingredient may be formulated in an aqueous pharmaceutically acceptable inhalant vehicle, such as, for example, isotonic saline or bacteriostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs.

Powder compositions containing anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule, which may be inserted by the patient into a device that punctures the capsule and blows the powder out in a steady stream.

Aerosol formulations for use in the subject method typically include propellants, surfactants, and co-solvents and may be filled into conventional aerosol containers that are closed by a suitable metering valve.

For oral administration, anti-inflammatory compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion or as a bolus, etc. Alternately, administration of a composition of all of the aspects of the present invention may be effected by liquid solutions, suspensions or elixirs, powders, lozenges, micronized particles and osmotic delivery systems.

Formulations of compositions of the present invention suitable for nasal administration, wherein the carrier is a solid, include a coarse powder having a particle size, for example, in the range of 20 to 500 microns which is administered in the manner in which snuff is administered, i.e. by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention. Semi-liquid formulations, such as a nasal gel, are also suitable.

Formulations of compositions suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents.

The pharmaceutical compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammal” or “mammal in need” include humans as well as non-human mammals, particularly domesticated animals including, without limitation, cats, dogs, and horses.

The term “therapeutically effective amount” is used to denote treatments at dosages effective to achieve the therapeutic result sought. Furthermore, one of skill will appreciate that the therapeutically effective amount of the compound of the invention may be lowered or increased by fine-tuning and/or by administering more than one compound of the invention, or by administering a compound of the invention with another ant-asthmatic compound (e.g., corticosteroid). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect. Clinical changes relevant to assess the therapeutic effect of treatment according to the invention include reduction in the characteristic symptoms and signs of asthma and related pathologies (e.g., dyspnea, wheezing, cough, bronchial hypersensitivity airway remodeling) and improvement of pulmonary function tests. These are based upon patient's symptoms and physician's observations.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

For local administration by inhalation for example, contemplated therapeutically effective amounts are from about 0.1 μg/kg/day to about 1000 μg/kg/day when administered systemically (e.g., orally administered). In an embodiment of the invention, when systemically administered, therapeutically effective amounts are from about 0.5 μg/kg/day to about 200 μg/kg/day.

Dosage forms and frequency of administration of the same, will depend on conventional factors routinely considered by one of skill in the field to obtain therapeutically effective amounts as discussed above in a given mammal. Hence, a practitioner will consider the condition being treated, the particular compound of the invention being administered, route of administration, and other clinical factors such as age, weight and condition of the mammal as well as convenience and patient compliance.

It will be appreciated by those of skill in the art that the number of administrations of the compounds according to the invention will vary from patient to patient based on the particular medical status of that patient at any given time.

When applicable (such as for the treatment of asthma, for example) the compound according to this aspect of the invention, may be administered prior to, at the same time, or after the mammal has been exposed to an antigen. In addition, the timing of the administration of the compound of the invention with relation to the exposure to an antigen will vary from mammal to mammal depending on the particular situation. A skilled practitioner will optimize administration by careful monitoring the patient while altering the timing and/or the order of administration of the compound of the invention. Hence, it will be understood that the mammal need not suffer from a pulmonary inflammation to benefit from the invention. The compounds of the invention may be administered prophylactically to individuals predisposed to develop asthma and/or an asthma-related pathology. For example, an individual allergic to pollen may be administered a compound of the invention (e.g., by oral administration) on a daily basis and/or prior to going to a pollen-rich area (e.g., a garden). Likewise, an individual with only a family history of asthmatic attacks may be administered the compounds of the invention prophylactically—to prevent or inhibit possible onset of such an asthmatic attack.

Based on the above facts the present invention also provides a method of treating acute and chronic inflammatory disorders of the airways of mammals—including asthma and asthma-related pathologies. This method comprises administering to a mammal in need of such treatment a therapeutically effective amount of a compound of formula (I)

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

Compounds of Formulas (VII)-(XIX)

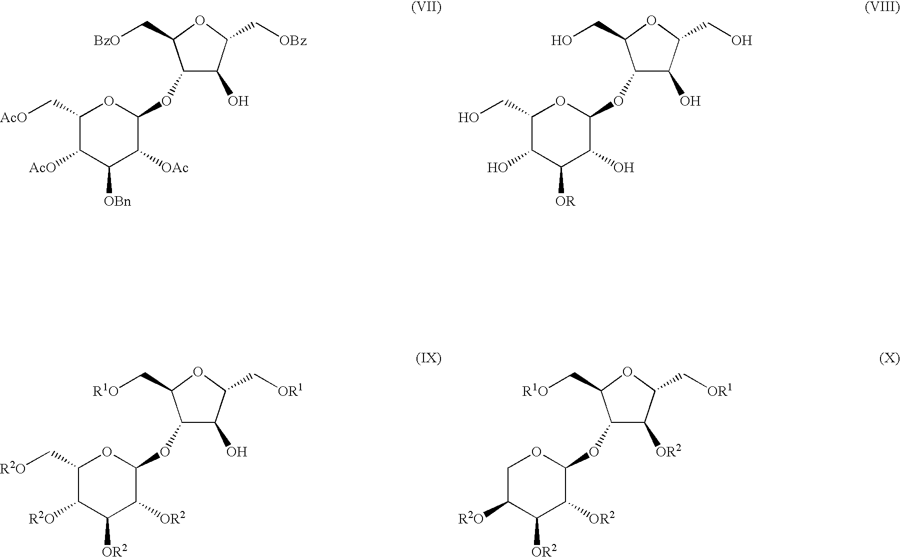

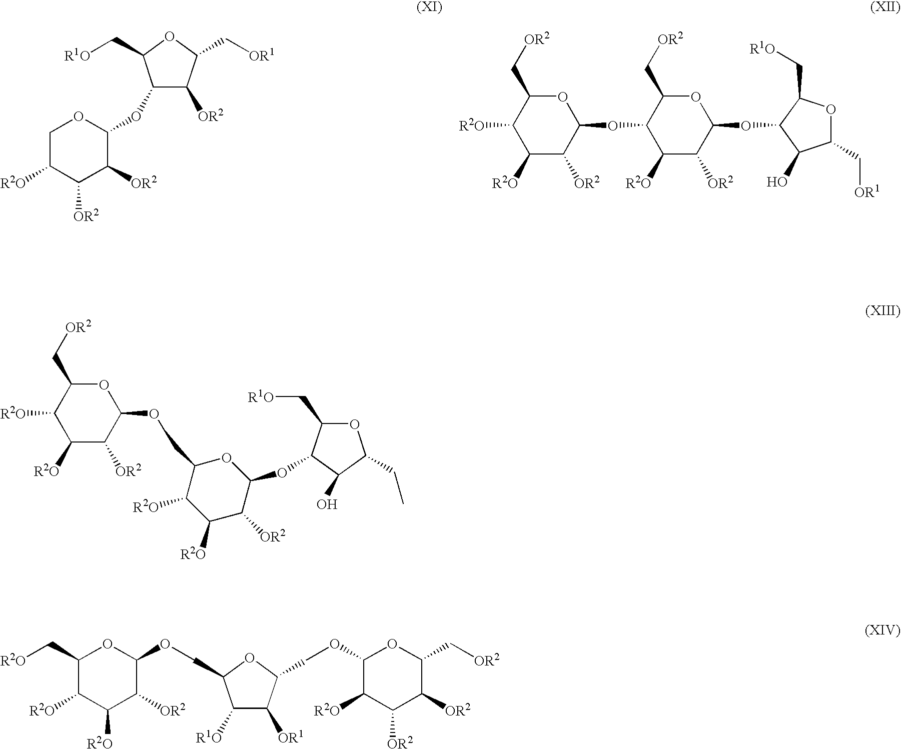

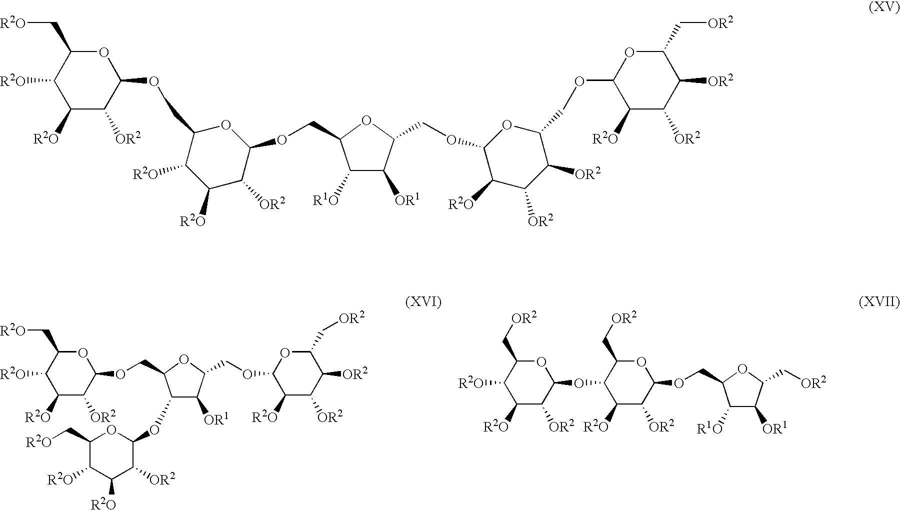

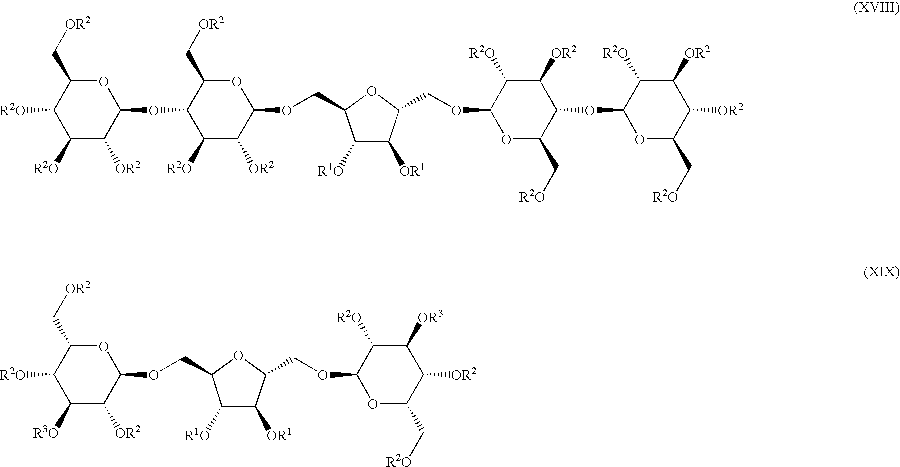

—wherein R as well as R¹ and R² stand for hydrogen—used as starting materials in the examples, are concrete, stereochemically well-defined, isomerically pure representatives of formula (VI).

The R_{f} values given in the examples were determined by thin layer chromatography using silica gel (DC-Alufolien Kieselgel 60 F254, Merck, Darmstadt) and the following mixtures of solvents:

(A) Ethyl acetate-hexane 1:1
(B) Ethyl acetate-hexane 1:2
(C) Ethyl acetate-hexane 1:3
(D) Ethyl acetate-hexane 2:1
(E) Ethyl acetate-ethanol 5:1

The spots were detected either in UV light or by spraying the plates with a 1:1 mixture of 0.1 M KMnO₄-1 M H₂SO₄ followed by heating to 200° C. Column chromatography was performed on Kieselgel 60. Optical rotations were measured at 20° C. NMR spectra were recorded with Bruker Avance 500 MHz spectrometer using Me₄Si as the internal standard. The assignments of the protons were based on COSY, 2D and selective 1D TOCSY as well as selective 1D NOESY experiments. Multiplicities of the ¹³C spectra were obtained from DEPT experiments. Connectivities between identified protons and protonated carbons were observed by means of HMQC and HMBC experiments.

In the case of acylation reactions carried out in the presence of pyridine the “usual work-up” means that if the product is not crystalline after pouring the reaction mixture into ice-water, it is extracted with an organic solvent, the organic layer is washed with water, 1 M ice-cold aqueous sulfuric acid solution until permanent acidity, water, 5% aqueous sodium bicarbonate solution and water, dried, filtered and the solvent is evaporated in vacuum.

### EXAMPLES

### Example 1

### 2,5-Anhydro-1,4,6-tri-O-sulfato-3-O-(2,3,4,6-tetra-O-sulfato-α-L-idopyranosyl)-D-mannitol hepta sodium salt (XX) (I, R¹=R³=R⁴=SO₃Na; R²=2,3,4,6-tetra-O-sulfato-α-L-idopyranosyl tetra sodium salt)

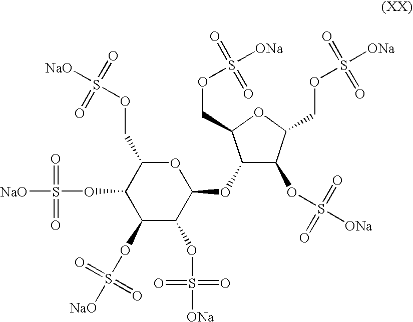

3.4 g (48%, 20 mmol) of sulfur trioxide-dimethylformamide complex was suspended in 5 ml of dry dimethylformamide with stirring, the mixture was cooled to −20° C. and 0.65 g (2 mmol) of glycoside of formula (VIII), R=H in 3 ml of dimethylformamide was gradually added at such a rate to keep the temperature below −15° C. After 15 min the temperature of the mixture was raised to −5° C. and kept there for 45 min. Thereafter the reaction mixture was again cooled to −20° C. and 1 ml of ethanol was gradually added at such a rate to keep the temperature below −15° C. Then the reaction mixture was poured into a stirred and cooled (−5° C.) solution of 4 g of sodium acetate and 30 ml of methanol. The precipitate was filtered off and washed with methanol. The solid residue is dissolved in 10 ml of water and the pH of the solution was adjusted first to 10 with 1 M sodium hydroxide solution, then to 5 with acetic acid. Thereafter 1 M aqueous strontium acetate solution was added to the solution until no more precipitate (SrSO₄) is formed. The precipitate was filtered off and the filtrate was submitted to a column loaded with CHELX 100 resin (sodium form) (10 mL) in order to remove strontium ions. The column was eluted with distilled water and the eluate was concentrated. The residue was treated with methanol, filtered and washed with methanol, the solid residue was suspended in 20 ml of methanol and stirred overnight at room temperature in order to remove sodium acetate. The crystals were filtered and washed with methanol to yield 1.64 g (79%) of the title compound; [α]_{D} −3° (c 1, water). NMR (D₂O) δ: ¹H, 5.22 (m, 1H, H-1′), 5.02 (m, 1H, H-3′), 4.86 (t, 1H, H-4), 4.45-4.60 (m, 6H, H-2,3,5,2′,4′,5′), 4.12-4.33 (m, 6H, H₂-1,6,1′); J_{3,4} ˜2.2, J_{4,5} ˜3.2, J_{1′,2′} ˜3, J_{2′,3′} ˜3, J_{3′,4′} ˜3 Hz. ¹³C, 100.0 (C-1′), 84.6, 84.4, 84.3, 83.2 (C-2,3,4,5), 73.4, 73.0, 72.7 (C-2′,3′,4′), 70.1, 69.8, 69.3 (C-1,6,6′), 66.8 (C-5′)

The starting material of formula (VIII) can be synthesized for example by the following method:

### Step a)

### Phenyl-2,4,6-tri-O-acetyl-3-O-benzyl-1-thio-L-idopyranoside (III, R⁸=R¹⁰=R¹¹=Ac, X=SPh)

To a stirred solution of 40 g (91 mmol) of crude tetraacetate (III, R⁸=R¹⁰=R¹¹=Ac, X=OAc) [C. A. A. van Boeckel, T. Beetz, J. N. Vos, A. J. M. de Jong, S. F. van Aelst, R. H. van den Bosch, J. M. R. Mertens, F. A. van der Vlugt, J. Carbohydr. Chem. 4 (1985) 293-321] in 500 ml of dichloromethane 11 ml (107 mmol) of thiophenol and 31 ml (245 mmol) of BF₃.Et₂O were added at 0° C. Stirring was continued at room temperature for 90 min, then the mixture was washed with 5% aqueous sodium bicarbonate solution and water, dried and concentrated to yield 46 g crude product, which was purified by column chromatography using eluent (C).

Concentration of the first fraction (R_{f} 0.6, solvent B) gave 5.6 g (13%) of β-anomer; [α]_{D} +27° (c 1, CHCl3).

Concentration of the second fraction (R_{f} 0.5, solvent B) gave 28 g (63%) of α-anomer; [α]_{D} −95° (c 1, CHCl₃). The 1H-NMR spectrum was identical with the one described in the literature [C. A. A. van Boeckel, T. Beetz, J. N. Vos, A. J. M. de Jong, S. F. van Aelst, R. H. van den Bosch, J. M. R. Mertens, F. A. van der Vlugt, J. Carbohydr. Chem. 4 (1985) 293-321].

### Step b)

### 2,5-Anhydro-3-O-(2,4,6-tri-O-acetyl-3-O-benzyl-α-L-idopyranosyl)-1,6-di-O-benzoyl-D-mannitol (VII)

To a stirred solution of 10 g (20 mmol) of thiophenyl glycoside (III, R⁸=R¹⁰=R¹¹=Ac, X=SPh) obtained in Step a) and 9.3 g (25 mmol) of 2,5-anhydro-1,6-dibenzoyl-D-mannitol (IV, R¹²=Bz; R¹³=H) [D. A. Otero and R. Simpson, Carbohydr. Res., 128 (1984) 79-86; N. Barroca and J-C. Jacquinet, Carbohydr. Res., 337 (2002) 673-689] in 200 ml of dichloromethane 30 g of freshly heated molecular sieves (4 Å) was added and the mixture was stirred at room temperature for 30 min. Then the reaction mixture was cooled to −40° C. and 6.9 g (1.5 equivalent) of NIS and 0.5 ml of TfOH were added. Stirring was continued at this temperature for 30 min, then the reaction was quenched by addition of 7 ml of Et3N. The reaction mixture was filtered, the filtrate was washed with aqueous sodium thiosulfate and sodium bicarbonate solution, dried and concentrated. The residue was purified by column chromatography (solvent A) to yield 10.85 g (71.5%) of the title compound; R_{f} 0.45, [α]_{D} −15° (c 1, CHCl₃).

### Step c)

### 2,5-Anhydro-3-O-(3-O-benzyl-α-L-idopyranosyl)-D-mannitol (VIII, R=Bn)

To a solution of 10.85 g (14.66 mmol) of the product obtained in the previous Step b) in 130 ml of methanol 2 ml of 2 M sodium methoxide solution in methanol was added. After 5 h, when according to TLC the deacylation reaction was complete (R_{f} 0.95→0.5, solvent E), sodium ions were removed by addition of cation exchange resin, the mixture was filtered and the filtrate was concentrated. The residue was purified by column chromatography (solvent E) to yield 5 g (82%) of the title compound as syrup. R_{f} 0.5, [α]_{D} −33° (c 1, water).

### Step d)

### 2,5-Anhydro-3-O-(α-L-idopyranosyl)-D-mannitol (VIII, R=H)

To a solution of 8.9 g (21.4 mmol) of the product obtained in the previous Step c) in 200 ml of methanol and 10 ml of water 0.3 ml of acetic acid and 1.5 g of 10% Pd/C catalyst were added. The reaction mixture was shaked in hydrogen atmosphere at room temperature for 6 h, when according to TLC the hydrogenolysis of the benzyl group was complete (R_{f} 0.5→0.1, solvent E). The reaction mixture was filtered, the filtrate was concentrated, the residue was treated with methanol, cooled and the obtained crystals were filtered and washed with cold methanol to yield 4.65 g (66%) of the title compound; Mp: 162-164° C., [α]_{D} −28° (c 1, water).

### Example 2

### 2,5-Anhydro-1,4,6-tri-O-sulfato-3-O-(2,3,4,6-tetra-O-sulfato-α-L-idopyranosyl)-D-mannitol hepta potassium salt (XXI) (I, R¹=R³=R⁴=SO₃K; R²=2,3,4,6-tetra-O-sulfato-α-L-idopyranosyl tetra potassium salt)

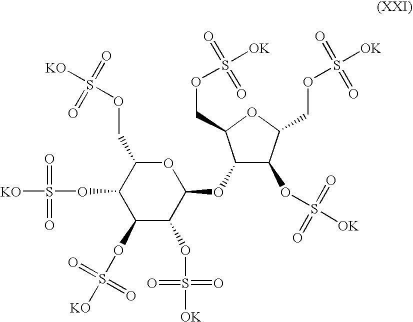

The title compound (XXI) was prepared according to the method described in Example 1, but the reaction mixture was poured into a stirred and cooled (0° C.) solution of potassium acetate in methanol and the pH of the solution of the filtered crude product was adjusted to 8 with 1 M potassium hydroxide. Yield: 89%, [α]_{D} −4° (c 1, water). C₁₂H₁₅O₃₁S₇K₇ Calculated: C, 12.50; H, 1.31; S, 16.46; K, 23.73. Found: C, 12.38; H, 1.82; S, 13.50; K, 22.90; Sr, 0.011. According to NMR spectra the sample contained 0.25 equivalent of potassium acetate and 0.07 equivalent of ethanol. NMR (D₂O) δ: ¹H, 5.23 (m, 1H, H-1′), 5.03 (m, 1H, H-3′), 4.86 (t, 0.1H, H-4), 4.46-4.60 (m, 6H, H-2,3,5,2′,4′,5′), 4.13-4.33 (m, 6H, H₂-1,6,1′); J_{3,4} ˜2.2, J_{4,5} ˜3.2, J_{1′,2′} ˜3, J_{2′,3′} ˜3, J_{3′,4′} ˜3 Hz. ¹³C, 100.1 (C-1′), 84.5, 84.4, 84.4, 83.2 (C-2,3,4,5), 73.3, 72.9, 72.8 (C-2′,3′,4′), 70.0, 69.7, 69.3 (C-1,6,6′), 66.7 (C-5′)

### Example 3

### 2,5-Anhydro-1,4,6-tri-O-sulfato-3-O-(2,3,4,6-tetra-O-sulfato-β-D-glucopyranosyl)-D-mannitol hepta sodium salt (XXII) (I, R¹=R³=R⁴=SO₃K; R²=2,3,4,6-tetra-O-sulfato-β-D-glucopyranosyl tetra sodium salt)

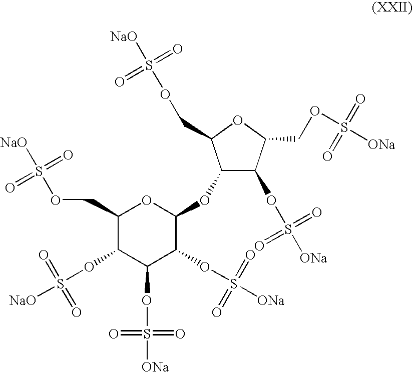

The title compound (XXII) was prepared according to the method described in Example 1 using the glycoside of formula (IX, R¹=R²=H) as starting material. Yield: 93%, [α]_{D} +15° (c 1, water). According to NMR spectra the sample contained 0.25 equivalent of sodium acetate and 0.25 equivalent of ethanol. NMR (D₂O) δ: ¹H, 4.88-4.94 (m, 2H, H-4,1′), 4.70 (m, 1H, H-3′), 4.32-4.55 (m, 6H, H-2,3,5,2′,4′,6′ₐ), 4.12-4.28 (m, 5H, H₂-1,6 és H-6′_{b}), 4.07 (m, 1H, H-5′); ¹³C, 102.1 (C-1′), 86.7, 84.5, 84.4, 83.6 (C-2,3,4,5), 79.5, 79.2, 76.4, 75.4 (C-2′,3′,4′,5′), 70.1, 69.7, 69.7 (C-1,6,6′).

The starting material of formula (IX, R¹=R²=H) can be synthesized for example by the following method:

### Step a)

### 3-O-(2,3,4,6-tetra-O-acetyl-β-D-glucopyranosyl)-2,5-anhydro-1,6-di-O-benzoyl-D-mannitol (IX, R¹=Bz, R²=Ac)

To a stirred solution of 9 g (24 mmol) of 2,5-anhydro-1,6-di-O-benzoyl-D-mannitol (IV, R¹²=Bz; R¹³=H) [D. A. Otero and R. Simpson, Carbohydr. Res., 128 (1984) 79-86; N. Barroca and J-C. Jacquinet, Carbohydr. Res., 337 (2002) 673-689] in 130 ml of acetonitrile 16 g of freshly heated molecular sieves (4 Å) was added and the mixture was stirred at room temperature for 30 min. Then 10.5 g (25.5 mmol) of acetobromoglucose and 6.3 g of Hg(CN)₂ were added and the mixture was stirred at room temperature for 20 h. Then the reaction mixture was diluted with 200 ml chloroform, washed with 5% aqueous sodium bicarbonate solution, 10% aqueous potassium bromide solution and water, dried and concentrated. The residue is purified by column chromatography (solvent A) to yield 5.4 g (30%) of the title compound; R_{f} 0.4; [α]_{D} +46° (c 1, CHCl₃).

### Step b)

### 2,5-Anhydro-3-O-(β-D-glucopyranosyl)-D-mannitol (IX, R¹=R²=H)

To a solution of 5.2 g (7.4 mmol) of the product obtained in the previous Step a) in 80 ml of methanol 0.6 ml of 2 M sodium methoxide solution in methanol was added at room temperature. After 5 h sodium ions were removed by addition of cation exchange resin, the mixture was filtered and the filtrate was concentrated. The residue was dissolved in water and extracted with chloroform in order to remove methyl benzoate. After concentration of the aqueous solution the residue was treated with ethanol and filtered to yield 1.7 g (71%) of the title compound; Mp 181-183° C., [α]_{D} +200 (c 1, water).

### Example 4

### 2,5-Anhydro-1,4,6-tri-O-sulfato-3-O-(2,3,4-tri-O-sulfato-α-L-arabinopyranosyl)-D-mannitol hexa potassium salt (XXIII) (I, R¹=R³=R⁴=SO₃K; R²=2,3,4-tri-O-sulfato-α-L-arabinopyranosyl tri potassium salt)

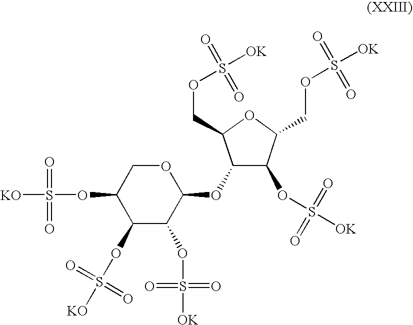

The title compound (XXIII) was prepared according to the method described in Example 2 using the glycoside of formula (X, R¹=R²=H) as starting material. Yield: 80%, [α]_{D} +33° (c 1, water). According to NMR spectra the sample contained 0.32 equivalent of potassium acetate and 0.28 equivalent of ethanol. C₁₁H₁₄O₂₇S₆K₆ Calculated: C, 13.14; H, 1.40; S, 19.14; K, 23.34. Found: C, 13.05; H, 1.78; S, 18.55; K, 23.30, Sr<0.01. NMR (D₂O) δ: ¹H, 5.02 (m, 1H, H-1′), 4.70-4.90 (m, 3H, H-4,2′,4′), 4.40-4.60 (m, 4H, H-2,3,5,3′), 4.17-4.33 (m, 4H, H₂-1,6), 3.78 (m, 1H) and 4.12 (m, 1H) (H, 5′); ¹³C, 100.8 (C-1′), 84.8, 84.2, 84.1, 82.8 (C-2,3,4,5), 76.6, 76.3, 74.2 (C-2′,3′,4′), 69.3, 69.7 (C-1,6), 63.3 (C-5′).

The starting material of formula (X, R¹=R²=H) can be synthesized for example by the following method:

### Step a)

### 3-O-(2,3,4,6-tetra-O-acetyl-α-L-arabinopyranosyl)-2,5-anhydro-1,4,6-tri-O-benzoyl-D-mannitol (X, R¹=Bz, R²=Ac)

To a stirred solution of 13 g (35 mmol) of 2,5-anhydro-1,6-di-O-benzoyl-D-mannitol (IV, R¹²=Bz; R¹³=H) [D. A. Otero and R. Simpson, Carbohydr. Res., 125 (1984) 79-86; N. Barroca and J-C. Jacquinet, Carbohydr. Res., 337 (2002) 673-689] in 200 ml of acetonitrile 30 g of freshly heated molecular sieves (4 Å) was added and the mixture was stirred at room temperature for 30 min. Then 12 g (35.4 mmol) of acetobromo-L-arabinose and 8.7 g of Hg(CN)₂ were added and the reaction mixture was stirred for 20 h. The reaction mixture was diluted with 200 ml of chloroform, washed with 5% aqueous sodium bicarbonate solution, 10% aqueous potassium bromide solution and water, dried and concentrated. The residue was dissolved in 100 ml of pyridine and 15 ml of benzoyl chloride was added. The reaction mixture was kept at room temperature for 2 h and the crude syrup obtained after usual work-up was purified by column chromatography (solvent B) to yield 11 g (39%) of the title compound; R_{f} 0.4; [α]_{D} +12° (c 1, CHCl₃).

### Step b)

### 2,5-Anhydro-3-O-(α-L-arabinopyranosyl)-D-mannitol (X, R¹=R²H)

To a solution of 10.9 g of the product obtained in the previous Step a) in 130 ml of methanol 1.5 ml of 2 M sodium methoxide solution in methanol was added at room temperature. After 3 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was treated with ethanol and filtered to yield 4.1 g (−100%) of the title compound as amorphous hygroscopic powder; [α]_{D} +67° (c 1, water).

### Example 5

### 2,5-Anhydro-1,4,6-tri-O-sulfato-3-O-(2,3,4-tri-O-sulfato-α-D-arabinopyranosyl)-D-mannitol hexa potassium salt (XXIV) (I, R¹=R³=R⁴=SO₃K; R²=2,3,4-tri-O-sulfato-α-D-arabinopyranosyl tri potassium salt)

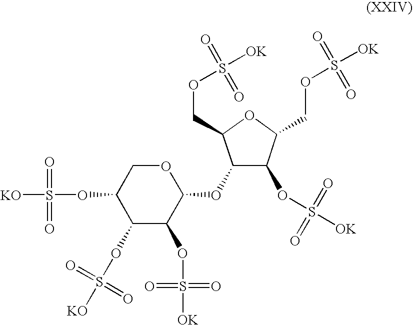

The title compound was prepared according to the method described in Example 2 using the appropriate D-arabinopyranoside of formula (XI, R¹=R²=H) as starting material. Yield: 95%, [α]_{D} +21° (c 1, water). According to NMR spectra the sample contained 0.30 equivalent of potassium acetate and 0.25 equivalent of ethanol. C₁₁H₁₄O₂₇S₆K₆ Calculated: C, 13.14; H, 1.40; S, 19.14; K, 23.34. Found: C, 13.00; H, 1.81; S, 18.65; K, 23.25; Sr<0.1. NMR (D₂O) δ: H, 4.88 (t, 1H, H-4), 4.81-4.86 (m, 2H, H-1′,4′), 4.68 (m, 1H, H-2′), 4.38-4.56 (m, 4H, H-2,3,5,3′), 4.15-4.30 (m, 4H, H2-1,6), 4.15 (m, 1H) and 3.75 (m, 1H) (H₂-5′); J_{3,4} ˜3, J_{4,5} ˜3, J_{5′gem} 12.6 Hz. ¹³C, 102.6 (C-1′), 86.2, 84.7, 84.0, 82.9 (C-2,3,4,5), 77.0, 76.6, 74.5 (C-2′,3′,4′), 70.0, 69.3 (C-1,6), 63.5 (C-5′)

The starting material of formula (XI, R¹=R²=H) can be synthesized for example by the following method:

### Step a)

### 3-O-(2,3,4,6-tetra-O-acetyl-α-D-arabinopyranosyl)-2,5-anhydro-1,4,6-tri-O-benzoyl-D-mannitol (XI, R¹=Bz, R²=Ac)

The title compound was prepared according to the method described in Step a) of Example 4 using acetobromo-D-arabinose as donor in the glycosylation reaction. The optical rotation of the obtained title compound is [α]_{D} 0° (c 1, CHCl3).

### Step b)

### 2,5-Anhydro-3-O-(α-D-arabinopyranosyl)-D-mannitol (XI, R¹=R²=H)

The title compound was prepared according to the method described in Step b) of Example 4 using the appropriate protected D-arabinopyranoside of formula (XI, R¹=Bz, R²=Ac) as starting material. The optical rotation of the obtained title compound is [α]_{D} +7° (c 1, water).

### Example 6

### 2,5-Anhydro-1,4,6-tri-O-sulfato-3-O-(2,3,6,2′,3′,4′,6′-hepta-O-sulfato-β-D-maltosyl)-D-mannitol deca potassium salt (XXV) (I, R¹=R³=R⁴=SO₃K; R²=2,3,6,2,3′,4′,6′-hepta-O-sulfato-β-D-maltosyl hepta potassium salt)

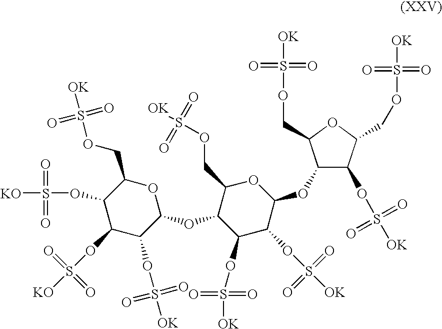

The title compound was prepared according to the method described in Example 2 using the glycoside of formula (XII, R¹=R²=H) as starting material. Yield: 95%, [α]_{D} +33° (c 1, water). C₁₈H₂₂O₄₅S₁₀K₁₀ Calculated: C, 12.95; H, 1.33; S, 19.20; K, 23.41. Found: C, 12.8; H, 1.65; S, 18.95; K, 23.08. NMR (D₂O) δ: ¹H, 5.58 (d, 1H, H-1″), 5.04 (d, 1H, H-1′), 4.95 (t, 1H, H-4), 4.86 (t, 1H, H-3′), 4.75 (t, 1H, H-2′), 4.15-4.60 (m, 16H, H-2,3,5,3′,5′2″,4″,5″ and H₂-1,6,6′,6″), 4.07 (m, 1H, H-4′); J_{3,4} 3.0, J_{4,5} 3.0, J_{1′,2′} 5.4, J_{2′,3′} 5.2, J_{1″,2″} 3.1, J_{2″,3″} 7.9, J_{2″,3″} 7.9 Hz. ¹³C, 103.7 (C-1′), 97.3 (C-1″), 86.8, 84.8, 84.4, 83.5 (C-2,3,4,5), 79.9, 79.4, 77.7, 76.5, 76.2, 76.1, 74.9, 72.7 (C-2′,3′,4′,5′,2″,3″,4″,5″), 70.2, 70.0, 69.7, 68.9 (C-1,6,6′,6″).

The starting material of formula (XII, R¹=R²=H) can be synthesized for example by the following method:

### Step a)

### 3-O-(hepta-O-acetyl-β-D-maltosyl)-2,5-anhydro-1,6-di-O-benzoyl-D-mannitol (XII, R¹=Bz, R²=Ac)

To a stirred solution of 6.5 (17.5 mmol) of 2,5-anhydro-1,6-di-O-benzoyl-D-mannitol (IV, R¹²=Bz; R¹³=H) [D. A. Otero and R. Simpson, Carbohydr. Res., 128 (1984) 79-86; N. Barroca and J-C. Jacquinet, Carbohydr. Res., 337 (2002) 673-689] in 120 ml of acetonitrile 18 g of freshly heated molecular sieves (4 Å) was added and the mixture was stirred at room temperature for 30 min. Then 12.5 g (18.4 mmol) of acetobromo-D-maltose and 5.5 g (19 mmol) of Hg(CN)₂ were added and the reaction mixture was stirred for 20 h. The reaction mixture was diluted with 300 ml of chloroform, washed with 5% aqueous sodium bicarbonate solution, 10% aqueous potassium bromide solution and water, dried and concentrated. The residue was purified by column chromatography (solvent A) to yield 5.6 g (30%) of the title compound; R_{f} 0.4; [α]_{D} +65° (c 1, CHCl₃).

### Step b)

### 2,5-Anhydro-3-O-(β-D-maltosyl)-D-mannitol (XII, R¹=R²=H)

To a solution of 1.6 g (1.6 mmol) of the product obtained in the previous Step a) in 20 ml of methanol 0.5 ml of 2 M sodium methoxide solution in methanol was added at room temperature. After 2 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was treated with ethanol and filtered to yield 0.75 g (95%) of the title compound as amorphous powder; [α]_{D} +95° (c 1, water).

### Example 7

### 2,5-Anhydro-1,4,6-tri-O-sulfato-3-O-[6-O-(2,3,4,6-tetra-O-sulfato-β-D-glucopyranosido)-2,3,4-tetra-O-sulfato-β-D-glucopyranosyl]-D-mannitol deca potassium salt (XXVI) (I, R¹=R³=R⁴=SO₃K; R²=6-O-(2,3,4,6-tetra-O-sulfato-β-D-glucopyranosido)-2,3,4-tetra-O-sulfato-β-D-glucopyranosyl hepta potassium salt

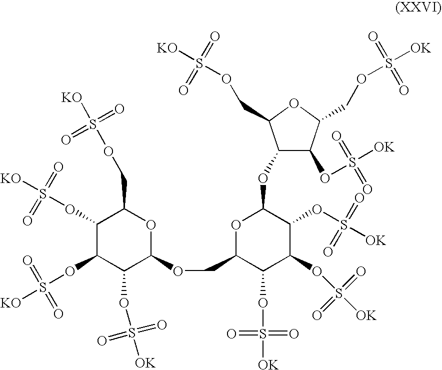

The title compound was prepared according to the method described in Example 2 using the glycoside of formula (XIII, R¹=R²=H) as starting material. Yield: 95%, [α]_{D} +7° (c 1, water). C₁₆H₂₂O₄₅S₁₀K₁₀ Calculated: C, 12.95; H, 1.33; S, 19.20; K, 23.41. Found: C, 12.48; H, 1.65; S, 18.95; K, 23.08. NMR (D₂O) δ: ¹H, 4.98 (d, 1H, H-1″), 4.91 (t, 1H, H-4), 4.83 (d, 1H, H-1′), 4.76 (t, 1H, H-4′), 4.68 (t, 1H, H-3″), 4.60 (t, 1H, H-3), 4.59 (t, 1H, H-3′), 4.50-4.55 (m, 2H, H-5 and Hₐ-6′), 4.44 (m, 1H, H-2), 4.40 (t, 1H, H-4″), 4.22-4.37 (m, 5H, H₂-1,6 and Hₐ-6′), 4.19 (dd, 1H, H_{b}-6″), 4.04 (m, 1H, H-5″), 4.02 (dd, 1H, H_{b}-6′), 3.90 (m, 1H, H-5′); J_{2,3} 3.3, J_{3,4} 3.9, J_{4,5} 4.0, J_{1′,2′} 7.4, J_{2′,3′} 8.5, J_{3′,4′} 8.7, J_{4′,6′} 8.7, J_{5′,6′a} 2.6, J_{5′,6′b} 3.8, J_{6′gem} 12.3, J_{1″,2″} 7.6, J_{2″,3″} 8.3, J_{3″,4″} 8.5, J_{4″,6″} 8.5, J_{5″,6″a} 2.6, J_{5″,6″b} 6.8, J_{6″gem} 11.2; ¹³C, 104.1 (C-1″), 103.1 (C-1′), 86.4 (C-3), 84.6 (C-4), 83.2 (C-5), 82.6 (C-2), 80.7 (C-3′), 79.9 (C-3″), 79.6 (C-2″), 79.3 (C-2′), 76.7 (C-5′), 76.4 (C-4″), 76.0 (C-4′), 75.5 (C-5″), 71.1 (C-6′), 70.1 (C-6″), 70.4 and 70.0 (C-1 and C-6)

The starting material of formula (XIII, R¹=R²=H) can be synthesized for example by the following method:

### Step a)

### 3-O-(hepta-O-acetyl-β-D-gentiobiosyl)-2,5-anhydro-1,6-di-O-benzoyl-D-mannitol (XII, R¹=Bz, R²=Ac)

A solution of 5 g (7.37 mmol) of octaacetyl-gentiobiose in 25 ml of 33% hydrogen bromide in acetic acid was stirred at 0° C. for 50 min. Then the reaction mixture was poured into ice-water, extracted with chloroform, the separated organic layer was washed with 5% aqueous sodium bicarbonate solution and water, dried and concentrated. 10 ml of toluene was evaporated from the residue to yield 4.7 g of crude acetobromo compound, which was used in the next reaction without further purification.

To a stirred solution of 2.6 g (7 mmol) of 2,5-anhydro-1,6-di-O-benzoyl-D-mannitol (IV, R¹²=Bz; R¹³=H) [D. A. Otero and R. Simpson, Carbohydr. Res., 128 (1984) 79-86; N. Barroca and J-C. Jacquinet, Carbohydr. Res., 337 (2002) 673-689] in 60 ml of acetonitrile 9 g of freshly heated molecular sieves (4 Å) was added and the mixture was stirred at room temperature for 30 min. Then 4.7 g (6.5 mmol) of freshly prepared acetobromo-gentiobiose obtained in the previous step and 2 g (7.9 mmol) of Hg(CN)₂ were added and the reaction mixture was stirred for 20 h. The reaction mixture was diluted with 200 ml of chloroform, washed with 5% aqueous sodium bicarbonate solution, 10% aqueous potassium bromide solution and water, dried and concentrated. The residue was treated with 30 ml of ethanol, filtered and recrystallized from 5-fold of ethanol to yield 2.2 g (32%) of the title compound; Mp: 166-168° C., [α]_{D} +9° (c 1, CHCl₃).

### Step b)

### 2,5-Anhydro-3-O-(β-D-gentiobiosyl)-D-mannitol (XIII, R¹=R²=H)

To a solution of 2 g (1.87 mmol) of the product obtained in the previous Step a) in 30 ml of methanol 0.3 ml of 2 M sodium methoxide solution in methanol was added at room temperature. After 3 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was treated with ethanol and filtered to yield 0.95 g (˜100%) of the title compound as amorphous powder; [α]_{D} +7° (c 1, water).

### Example 8

### 2,5-Anhydro-3,4-di-O-sulfato-1,6-bis-O-(2,3,4,6-tetra-O-sulfato-β-D-glucopyranosyl)-D-mannitol deca potassium salt (XXVII) (I, R¹=R⁴=2,3,4,6-tetra-O-sulfato-β-D-glucopyranosyl tetra potassium salt, R³=R⁴=SO₃K)

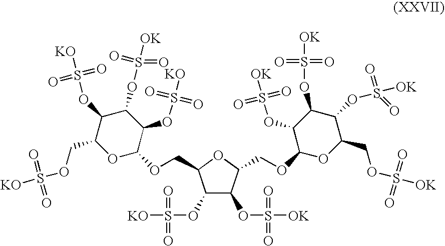

The title compound (XXVII) was prepared according to the method described in Example 2 using the glycoside of formula (XIV, R¹=R²=H) as starting material. Yield: 100%, [α]_{D} +33° (c 1, water). C₁₈H₂₂O₄₅S₁₀K₁₀ Calculated: C, 12.95; H, 1.33; S, 19.20; K, 23.41. Found: C, 12.40; H, 1.67; S, 18.87; K, 22.92. NMR (D₂O) δ: ¹H, 5.00-5.06 (m, 2H, H-3,4), 4.92 (d, 2H, H-1′), 4.74 (t, 2H, H-3′), 4.35-4.55 (m, 8H, H-2,5,2′,4′,6′ₐ), 4.13-4.27 (m, 4H, H-1ₐ,6ₐ,6′_{b}), 4.1 (m, 2H, H-5′), 3.91 (m, 2H, H-1_{b},6_{b}), J_{1′,2′} 5.6 Hz. ¹³C, 103.7 (C-1′), 83.2 (C-3,4), 83.0 (C-2,5) 78.9 (C-2′), 78.9 C-3′), 76.2 (C-5′), 75.6 (C.4′) 71.6 (C-1.6), 70.3 (C-6′)

The starting material of formula (XIV, R¹=R²=H) can be synthesized for example by the following method:

### Step a)

### 2,5-Anhydro-3,4-di-O-benzoyl-D-mannitol (IV, R¹²=H, R³=Bz)

To a stirred solution of 13 g (20 mmol) of 2,5-anhydro-1,6-di-O-trityl-D-mannitol (IV, R¹²=Tr; R¹³=H) [G. O. Aspinall et al., Carbohydrate Res., 66 (1978) 225-243] in 60 ml of pyridine 6 ml (50 mmol) of benzoyl chloride was added at 10° C. The reaction mixture was stirred at this temperature for 3 h, then worked up the usual way to yield 17 g (˜100%) of the crude product (IV, R¹²=Tr, R¹³=Bz), which was dissolved in 100 ml of acetic acid and 25 ml of water and stirred at 80° C. for 1 h. After cooling the precipitated trityl alcohol was filtered off, the filtrate was diluted with chloroform, washed with water and 5% aqueous sodium bicarbonate solution, dried and concentrated. The semisolid residue was treated with toluene, filtered and recrystallized from 3-fold of toluene to yield 2.6 g (35%) of the title compound; Mp: 120-122° C.

### Step b)

### 1,6-Bis-O-(2,3,4,6-tetra-O-acetyl-β-D-glucopyranosyl)-2,5-anhydro-3,4-di-O-benzoyl-D-mannitol (XIV, R¹=Bz, R²=Ac)

To a stirred solution of 2.6 g (7 mmol) of 2,5-anhydro-3,4-di-O-benzoyl-D-mannitol (IV, R¹²=H; R¹³=Bz) obtained in the previous Step a) in 65 ml of acetonitrile 9 g of freshly heated molecular sieves (4 Å) was added and the mixture was stirred at room temperature for 30 min. Then 5.8 g (14 mmol) of acetobromo-glucose and 4 g (15.8 mmol) of Hg(CN)₂ were added and the reaction mixture was stirred for 20 h. The reaction mixture was diluted with 200 ml of chloroform, washed with 5% aqueous sodium bicarbonate solution, 10% aqueous potassium bromide solution and water, dried and concentrated. The residue was purified by column chromatography (solvent D) to yield 2.0 g (28%) of the title compound; Mp: 132-134° C.; R_{f} 0.8; [α]_{D} −28° (c 1, CHCl₃).

### Step c)

### 2,5-Anhydro-1,6-bis-β-D-glucopyranosyl-D-mannitol (XIV, R¹=R²=H)

To a solution of 1.6 g (1.9 mmol) of the product obtained in the previous Step b) in 30 ml of methanol 0.3 ml of 2 M sodium methoxide solution in methanol was added at room temperature. After 2 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was treated with ethanol and filtered to yield 0.95 g (˜100%) of the title compound as amorphous powder; [α]_{D} −7° (c 1, water).

### Example 9

### 2,5-Anhydro-3,4-di-O-sulfato-1,6-bis-[(6-O-2,3,4,6-tetra-O-sulfato-β-D-glucopyranosido)-2,3,4-tri-O-sulfato-β-D-glucopyranosyl]-D-mannitol hexadeca potassium salt (XXVIII) (I, R¹=R⁴=[(6-O-2,3,4,6-tetra-O-sulfato-β-D-glucopyranosido)-2,3,4-tri-O-sulfato-β-D-glucopyranosyl]hepta potassium salt, R²=R³=SO₃K)

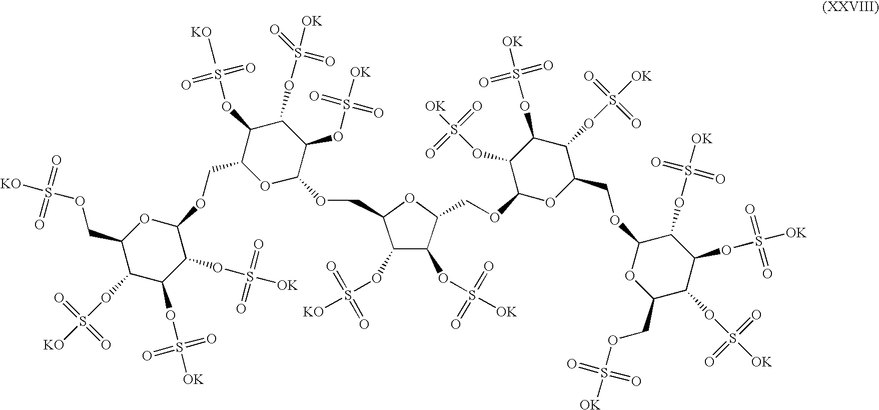

The title compound (XXVIII) was prepared according to the method described in Example 2 using the glycoside of formula (XV, R¹=R²=H) as starting material. Yield: 82%, [α]_{D} 0° (c 1, water). C₃₀H₃₆O₇₃S₁₆K₁₆ Calculated: C, 13.33; H, 1.33; S, 18.96; K, 23.10. Found: C, 12.62; H, 1.82; S, 17.48; K, 21.24. NMR (D₂O) δ: ¹H, 5.00 (m, 2H, H-3,4), 4.87 (d, 2H) and 4.83 (d, 2H) (H1′ and H-1″), 4.15-4.65 (m, 22H) and 3.90-4.05 (m, 8H) (H-2,5,2′,3′,4′,5′,2″,3″,4″,5″, H₂-1,6,1′,1″); J_{1′,2′} 7.3, J_{1″,2″} 7.3 Hz. ¹³C, 104.0, 103.9 (C-1′,1″), 83.0, 82.9 (C-2,3,4,5), 80.3, 79.9, 79.1, 79.1, 76.2, 76.2, 76.0, 75.6 (C-2′,3′,4′,5′,2″,3″,4″,5″), 72.2, 71.6, 70.4 (C-1,6,6′,6″)

The starting material of formula (XV, R¹=R²=H) can be synthesized for example by the following method:

### Step a)

### 2,5-Anhydro-3,4-di-O-benzoyl-1,6-bis-[(6-O-2,3,4,6-tetra-O-acetyl-β-D-glucopyranosido)-2,3,4-tri-O-acetyl-β-D-glucopyranosyl]-D-mannitol (XV, R¹=Bz, R²=Ac)

To a stirred solution of 1 g (2.7 mmol) of 2,5-anhydro-3,4-di-O-benzoyl-D-mannitol (IV, R¹²=H; R¹³=Bz), obtained according to the method described in Step a) of Example 8, in 50 ml of acetonitrile 5 g of freshly heated molecular sieves (4 Å) was added and the mixture was stirred at room temperature for 30 min. Then 4.16 g (6 mmol) of acetobromo-gentiobiose and 1.65 g (6.5 mmol) of Hg(CN)₂ were added and the reaction mixture was stirred for 20 h. The reaction mixture was diluted with 300 ml of chloroform, washed with 5% aqueous sodium bicarbonate solution, 10% aqueous potassium bromide solution and water, dried and concentrated. The residue was purified by column chromatography (solvent D) to yield 2.1 g (48%) of the title compound; R_{f} 0.4; [α]_{D} −17° (c 1, CHCl₃).

### Step b)

### 2,5-Anhydro-1,6-bis-(6-O-β-D-glucopyranosido-β-D-glucopyranosyl)-D-mannitol (XV, R¹=R²=H)

To a solution of 2 g (1.24 mmol) of the product obtained in the previous Step a) in 30 ml of methanol 0.3 ml of 2 M sodium methoxide solution in methanol was added at room temperature. After 3 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was treated with ethanol and filtered to yield 0.8 g (80%) of the title compound as amorphous powder; [α]_{D} −8° (c 1, water).

### Example 10

### 2,5-Anhydro-4-O-sulfato-1,3,6-tris-O-(2,3,4,6-tetra-O-sulfato-β-D-glucopyranosyl)-D-mannitol trideca potassium salt (XXIX) (I, R¹=R²=R⁴=2,3,4,6-tetra-O-sulfato-β-D-glucopyranosyl tetra potassium salt, R³=SO₃K)

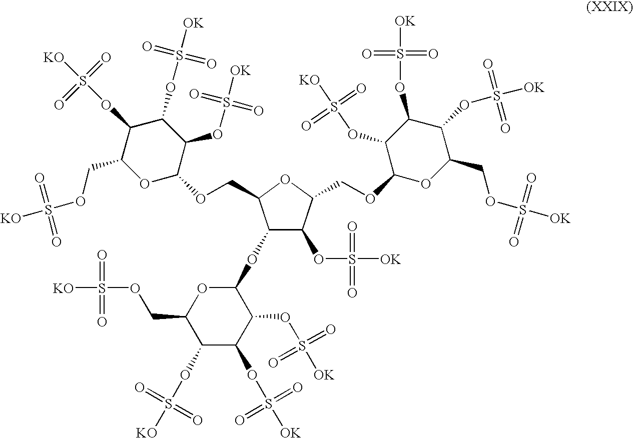

The title compound (XXIX) was prepared according to the method described in Example 2 using the glycoside of formula (XVI, R¹=R²=H) as starting material. Yield: 69%, [α]_{D} +4° (c 1, water). C₂₄H₂₉O₅₉S₁₃K₁₃ Calculated: C, 13.18; H, 1.34; S, 19.06; K, 23.25. Found: C, 12.38; H, 1.77; S, 18.68; K, 21.85. NMR (D₂O) δ: ¹H, 3.90-5.05 (m, 32H); ¹³C, 103.8 (C-11,1″,1′″), 85.9, 84.3, 84.2, 82.0 (C-2,3,4,5), 79.4, 79.4, 79.3, 79.1, 78.9, 78.9, 76.3, 76.2, 76.0, 75.9, 75.8, 75.5 (C-2′,3′,4′,5′,2″,3″,4″,5″,2′″,3′″,4′″,5′″), 72.7, 70.7, 70.2, 70.1, 69.7 (C-1,6,6′,6″,6′″).

The starting material of formula (XVI, R¹=R²=H) can be synthesized for example by the following method:

### Step a)

### 2,5-Anhydro-1,3,6-tris-2,3,4,6-tetra-O-acetyl-β-D-glucopyranosido-D-mannitol (XVI, R¹=H, R²=Ac)

To a stirred solution of 1.64 g (10 mmol) of 2,5-anhydro-D-mannitol ((IV, R¹²=R¹³=H) [D. A. Otero and R. Simpson, Carbohydr. Res., 128 (1984) 79-86] in 100 ml of acetonitrile 12 g of freshly heated molecular sieves (4 Å) was added and the mixture was stirred at room temperature for 30 min. Then 14.8 g (36 mmol) of acetobromo-glucose and 10 g (33.7 mmol) of Hg(CN)₂ were added and the reaction mixture was stirred for 20 h. The reaction mixture was diluted with 300 ml of chloroform, washed with 5% aqueous sodium bicarbonate solution, 10% aqueous potassium bromide solution and water, dried and concentrated. The residue was purified by column chromatography (solvent E) to yield 3.9 g (34%) of the title compound; R_{f} 0.4; [α]_{D} +4° (c 1, CHCl₃).

### Step b)

### 2,5-Anhydro-1,3,6-tris-β-D-glucopyranosyl-D-mannitol (XVI, R¹=R²=H)

To a solution of 3.7 g (3.2 mmol) of the product obtained in the previous Step a) in 30 ml of methanol 0.3 ml of 2 M sodium methoxide solution in methanol was added at room temperature. After 3 h sodium ions were removed by addition of cation exchange resin, the mixture was filtered and the filtrate was freeze-dried to yield 1.8 g (97%) of the title compound as amorphous powder; [α]_{D} +8° (c 1, water).

### Example 11

### 2,5-Anhydro-3,4,6-tri-O-sulfato-1-O-(2′,3′,4′,6′,2,3,6-hepta-O-sulfato-β-D-maltosyl)-D-mannitol deca potassium salt (XXX) (I, R¹=2′,3′,4′,6′,2,3,6-hepta-O-sulfato-β-D-maltosyl hepta potassium salt, R²=R³=R⁴=SO₃K)

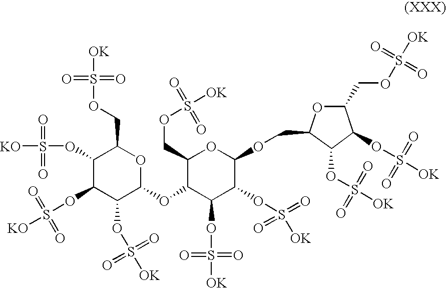

The title compound (XXX) was prepared according to the method described in Example 2 using the glycoside of formula (XVII, R¹=R²=H) as starting material. Yield: 90%, [α]_{D} +31° (c 1, water). C₁₈H₂₂O₄₅S₁₀K₁₀ Calculated: C, 12.95; H, 1.33; S, 19.20; K, 23.41. Found: C, 12.63; H, 1.68; S, 18.30; K, 22.96. NMR (D₂O) δ: ¹H, 5.56 (m, 1H, H-1″), 5.02 (m, 1H, H-1′), 4.98 (m, 1H, H-3), 4.95 (m, 1H, H-4), 4.76-4.84 (m, 2H, H-3′ and H-3″), 4.55 (m, 1H, H-2′), 4.47-4.52 (m, 2H, H-5 and H-2″), 4.40-4.46 (m, 3H, H-2,4″ and Hₐ-6′), 4.20-4.35 (m, 6H, H₂-6, H₂-6″, H-4′ and H_{b}-6′), 4.05-4.20 (m, 3H, H-5′,5″ and Hₐ-1), 3.89 (m, 1H, H_{b}-1). ¹³C, 104.0 (C-1′), 97.4 (C-1″), 84.1, 84.0, 83.8, 83.0 (C-2,3,4,5), 79.1 (C-3′), 78.7 (C-2′), 77.8 (C-3″), 76.6 (C-2″), 76.4 (C.4″), 75.9 (C-5′), 74.9 (C-4′), 72.7 (C-5″), 71.3 (C-1), 70.4 (C-6′), 70.0 (C-6), 69.0 (C-6″)

The starting material of formula (XVII, R¹=R²=H) can be synthesized for example by the following method:

### Step a)

### 6-O-Acetyl-2,5-anhydro-3,4-di-O-benzoyl-1-O-(2′,3′,4′,6′,2,3,4-hepta-O-acetyl-β-D-maltosyl)-D-mannitol (XVII, R¹=Bz, R²=Ac)

To a stirred solution of 1.6 g (4.3 mmol) of 2,5-anhydro-3,4-di-O-benzoyl-D-mannitol (IV, R¹²=H; R¹³=Bz), obtained according to the method described in Step a) of Example 8, in 60 ml of dry dichloromethane 6 g of freshly heated molecular sieves (4 Å) and 7 g (9.6 mmol) of phenyl-β-thiomaltoside peracetate (III, R⁸=R⁹=R¹¹=Ac; R¹⁰=pentaacetyl-α-D-glucopyranoside, X=SPh) [A. J. Pearce et al., Eur. J. Org. Chem. 9 (1999) 2103-2118; M. Cudic et al., Biorg. Med. Chem. 10 (2002) 3859-3870] were added and the mixture was stirred at room temperature for 30 min. Then the reaction mixture was cooled to −40° C. and 3.15 g (14 mmol) of NIS and 0.2 ml of TfOH were added and stirring was continued at −40° C. for 25 min. Thereafter 3 ml of triethylamine was added to the reaction mixture and the temperature was allowed to raise to room temperature. The mixture was filtered, the filtrate was washed with aqueous sodium thiosulfate solution, sodium bicarbonate solution and water, dried and concentrated. The residue was purified by column chromatography (solvent D) to yield 1.5 g (33%) of the title compound; R_{f} 0.7; [α]_{D} +100 (c 1, CHCl₃).

### Step b)

### 2,5-Anhydro-1-O-β-maltopyranosyl-D-mannitol (XVII, R¹=R²=H)

To a solution of 1.3 g (1.2 mmol) of the product obtained in the previous Step a) in 15 ml of methanol 0.2 ml of 2 M sodium methoxide solution in methanol was added at room temperature. After 3 h sodium ions were removed by addition of cation exchange resin, the mixture was filtered and the filtrate was concentrated. The residue was dissolved in water and extracted with chloroform in order to remove methyl benzoate. Freeze-drying of the aqueous solution yielded 0.6 g (60%) of the title compound as amorphous powder; [α]_{D} +86° (c 1, water).

### Example 12

### 2,5-Anhydro-3,4-di-O-sulfato-1,6-bis-O-(2′,3′,4′,6′,2,3,6-hepta-O-sulfato-β-D-maltosyl)-D-mannitol hexadeca potassium salt (XXXI) (I, R¹=R⁴2′,3′,4′,6′,2,3,6-hepta-O-sulfato-β-D-maltosyl hepta potassium salt, R¹=R³=SO₃K)

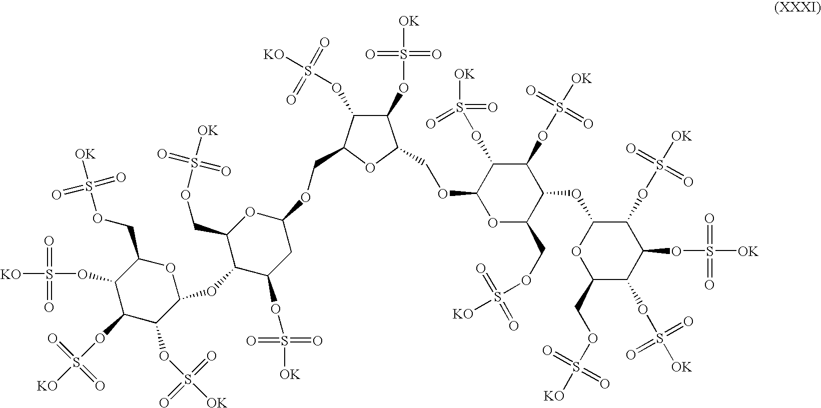

The title compound (XXXI) was prepared according to the method described in Example 2 using the glycoside of formula (XVIII, R¹=R²=H) as starting material. Yield: 71%, [α]_{D} +35° (c 1, water). C₃₀H₃₆O₇₃S₁₆K₁₆ Calculated: C, 13.33; H, 1.33; S, 18.96; K, 23.10. Found: C, 12.85; H, 1.72; S, 18.57; K, 22.52. NMR (D₂O) δ: ¹H, 5.57 (m, 2H, H-1″), 5.03 (m, 4H, H-3,4,1′), 4.77-4.87 (m, 4H, H-3′,3″), 4.50-4.55 (m, 4H, H-2′,2″), 4.40-4.50 (m, 6H, H-2,5,4″ and Hₐ-6″), 4.28-4.35 (m, 6H, H₂-6′ and H_{b}-6′), 4.25 (m, 2H, H-4′), 4.10-4.21 (m, 6H, H-5′,5″ and Hₐ-1,6), 3.91 (m, 2H, H_{b}-1,6). ¹³C, 104.1 (C-1′), 97.5 (C-1″), 83.2 (C-3,4), 83.0 (C-2,5), 79.4 (C-3′), 78.9 (C-2′), 77.7 (C-3″), 76.5 (C-2″), 76.2 (C.4″), 76.1 (C-5′), 75.1 (C-4′), 72.7 (C-5″), 71.4 (C-1,6), 70.3 (C-6′), 69.0 (C-6″)

The starting material of formula (XVIII, R¹=R²=H) can be synthesized for example by the following method:

### Step a)

### 2,5-Anhydro-3,4-di-O-benzoyl-1,6-bis-O-(2′,3′,4′,6′,2,3,6-hepta-O-acetyl-β-D-maltosyl)-D-mannitol (XVIII, R¹=Bz, R²=Ac)

To a stirred solution of 2.75 g (7.4 mmol) of 2,5-anhydro-3,4-di-O-benzoyl-D-mannitol (IV, R¹²=H; R¹³=Bz), obtained according to the method described in Step a) of Example 8, in 150 ml of acetonitrile 18 g of freshly heated molecular sieves (4 Å) was added and the mixture was stirred at room temperature for 30 min. Then 14 g (20 mmol) of acetobromo-D-maltose and 5.5 g of Hg(CN)₂ were added and the reaction mixture was stirred for 20 h. The reaction mixture was diluted with 300 ml of chloroform, washed with 5% aqueous sodium bicarbonate solution, 10% aqueous potassium bromide solution and water, dried and concentrated. The residue was purified by column chromatography (solvent D) to yield 6.3 g (48%) of the title compound; R_{f} 0.35; [α]_{D} +38° (c 1, CHCl₃).

### Step b)

### 2,5-Anhydro-1,6-bis-O-β-maltopyranosyl-D-mannitol (XVIII, R¹=R²=H)

To a solution of 6.3 g (3.9 mmol) of the product obtained in the previous Step a) in 100 ml of methanol 1 ml of 2 M sodium methoxide solution in methanol was added at room temperature. After 3 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was purified by column chromatography using a 1:5 mixture of ethyl acetate:methanol as eluent. Concentration of the proper fractions (R_{f} 0.4) yielded 0.7 g (22%) of the title compound; [α]_{D} +92° (c 1, water).

### Example 13

### 2,5-Anhydro-3,4-di-O-sulfato-1,6-bis-O-(2,3,4,6-tetra-O-sulfato-α-L-idopyranosyl)-D-mannitol deca potassium salt (XXXII) (I, R¹=R⁴=2,3,4,6-tetra-O-sulfato-α-L-idopyranosyl tetra potassium salt, R³=R⁴=SO₃K)

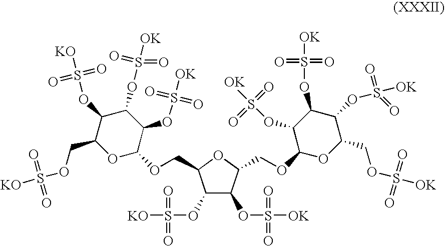

The title compound (XXXII) was prepared according to the method described in Example 2 using the glycoside of formula (XIX, R¹=R²=R³=H) as starting material. Yield: 75%, [α]_{D} −13° (c 1, water). C₁₈H₂₂O₄₅S₁₀K₁₀ Calculated: C, 12.95; H, 1.33; S, 19.20; K, 23.41. Found: C, 12.48; H, 1.62; S, 18.89; K, 22.71. NMR (D₂O) δ: ¹H, 5.57 (m, 2H, H-1″), 5.03 (m, 4H, H-3,4,1′), 4.77-4.87 (m, 4H, H-3′,3″), 4.50-4.55 (m, 4H, H-2′,2″), 4.40-4.50 (m, 6H, H-2,5,4″ and Hₐ-6″), 4.28-4.35 (m, 6H, H₂-6′ and H_{b}-6″), 4.25 (m, 2H, H-4′), 4.10-4.21 (m, 6H, H-5′,5″ and Hₐ-1,6), 3.91 (m, 2H, H_{b}-1,6). ¹³C, 104.1 (C-1′), 97.5 (C-1″), 83.2 (C-3,4), 83.0 (C-2,5), 79.4 (C-3′), 78.9 (C-2′), 77.7 (C-3″), 76.5 (C-2″), 76.2 (C-4″), 76.1 (C-5′), 75.1 (C-4′), 72.7 (C-5″), 71.4 (C-1,6), 70.3 (C-6′), 69.0 (C-6″).

The starting material of formula (XIX, R¹=R²=R³=H) can be synthesized for example by the following method:

### Step a)

### 1,6-Bis-O-(2,4,6-tri-O-acetyl-3-O-benzyl-α-L-idopyranosyl)-2,5-anhydro-3,4-di-O-benzoyl-D-mannitol (XIX, R¹=Bz, R²=Ac, R³=Bn)

To a stirred solution of 4.46 g (12 mmol) of 2,5-anhydro-3,4-di-O-benzoyl-D-mannitol (IV, R¹²=H; R¹³=Bz), obtained according to the method described in Step a) of Example 8, in 150 ml of dry dichloromethane 18 g of freshly heated molecular sieves (4 Å) was added and the mixture was stirred at room temperature for 30 min. Then 13 g (27 mmol) of phenyl-2,4,6-tri-O-acetyl-3-O-benzyl-1-thio-α-L-idopyranoside [Ch. Tabeur et al., Carbohydrate Res., 281 (1996) 253-276] was added. The reaction mixture was cooled to −40° C., 9 g (40 mmol) of NIS and 0.5 ml of TfOH were added and stirring was continued at −40° C. for 15 min. Thereafter 9 ml of triethylamine was added to the reaction mixture and the temperature was allowed to raise to room temperature. The mixture was filtered, the filtrate was washed with aqueous sodium thiosulfate solution, sodium bicarbonate solution and water, dried and concentrated. The residue was purified by column chromatography (solvent A) to yield 8.0 g (59%) of the title compound; R_{f} 0.35; [α]_{D} −75° (c 1, CHCl₃). According to NMR spectra the purity of the product was ˜60%, but it could be used in the next step without further purification.

### Step b)

### 2,5-Anhydro-1,6-bis-(3-O-benzyl-α-L-idopyranosyl)-D-mannitol (XIX, R¹=R²=H, R³=Bn)

To a solution of 8 g (7.1 mmol) of the product obtained in the previous Step a) in 80 ml of methanol 0.3 ml of 2 M sodium methoxide solution in methanol was added at room temperature. After 2 h sodium ions were removed by addition of cation exchange resin, the mixture was filtered and the filtrate was concentrated. The residue was dissolved in water and extracted with chloroform in order to remove methyl benzoate. After freeze-drying of the aqueous solution the residue was purified by column chromatography (solvent E) to yield 1.7 g (36%) of the title compound; R_{f} 0.45 (solvent E); [α]_{D} −41° (c 1, water).

### Step c)

### 2,5-Anhydro-1,6-bis-α-L-idopyranosyl-D-mannitol (XIX, R¹=R²=R³=H)

To a stirred solution of 1.7 g of the benzyl derivative obtained in the previous Step b) in 50 ml of methanol and 5 ml of water 1 g of 10% Pd/C catalyst was added and the mixture was hydrogenated at atmospheric pressure for 6 h. When according to TLC the cleavage of the benzyl groups was complete, the catalyst was filtered off, the filtrate was concentrated, the residue was dissolved in 10 ml of water and freeze-dried to yield 1.2 g (96%) of the title compound; [α]_{D} 0° (c 1, water).

### EQUIVALENTS

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What we claim is:

1. A compound of formula (I), 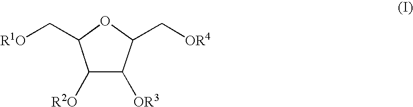
wherein R¹, R², R³ and R⁴ are independently selected from the groups consisting of H, —SO₃H, a sulfated or unsulfated glycosyl group or a sulfated or unsulfated diglycosyl group with the proviso that at least one of R¹-R⁴ is a sulfated or unsulfated glycosyl group or a sulfated or unsulfated diglycosyl group—
a pharmaceutically acceptable salt or isomer thereof.

2. A compound of formula (I) according to claim 1 wherein R¹, R³ and R⁴ are selected from —SO₃H and R² is a sulfated glycosyl group or alkali or alkaline earth metal salt or isomer thereof.

3. A compound of formula (I) according to claim 1, wherein R¹, R³ and R⁴ are selected from —SO₃H and R² represents a sulfated diglycosyl group or alkali or alkaline earth metal salt or isomer thereof.

4. A compound of formula (I) according to claim 1, wherein R¹ and R⁴ are independently selected from a sulfated glycosyl or diglycosyl group and R² and R³ is —SO₃H or an alkali or alkaline earth metal salt or isomer thereof.

5. A compound of formula (I) according to claim 1 wherein R¹, R² and R⁴ are selected from a sulfated glycosyl group and R³ is —SO₃H or an alkali or alkaline earth metal salt or isomer thereof.

6. A compound of formula (I) according to claim 1 selected from the group consisting of
2,5-Anhydro-1,4,6-tri-O-sulfato-3-O-(2,3,4,6-tetra-O-sulfato-α-L-idopyranosyl)-D-mannitol; 2,5-Anhydro-1,4,6-tri-O-sulfato-3-O-(2,3,4,6-tetra-O-sulfato-β-D-glucopyranosyl)-D-mannitol; 2,5-Anhydro-1,4,6-tri-O-sulfato-3-O-(2,3,4-tri-O-sulfato-α-L-arabinopyranosyl)-D-mannitol; 2,5-Anhydro-1,4,6-tri-O-sulfato-3-O-(2,3,4-tri-O-sulfato-α-D-arabinopyranosyl)-D-mannitol; 2,5-Anhydro-1,4,6-tri-O-sulfato-3-O-(2′,3′,4′,6′,2,3,6-hepta-O-sulfato-β-D-maltosyl)-D-mannitol; 2,5-Anhydro-1,4,6-tri-O-sulfato-3-O-[6-O-(2,3,4,6-tetra-O-sulfato-β-D-glucopyranosido)-2,3,4-tetra-O-sulfato-β-D-glucopyranosyl]-D-mannitol;
2,5-Anhydro-3,4-di-O-sulfato-1,6-bis-O-(2,3,4,6-tetra-O-sulfato-β-D-glucopyranosyl)-D-mannitol; 2,5-Anhydro-3,4-di-O-sulfato-1,6-bis-[(6-O-2,3,4,6-tetra-O-sulfato-β-D-glucopyranosido)-2,3,4-tri-O-sulfato-β-D-glucopyranosyl]-D-mannitol;
2,5-Anhydro-4-O-sulfato-1,3,6-tri-O-[2,3,4,6-tetra-O-sulfato-β-D-glucopyranosyl]-D-mannitol; 2,5-Anhydro-3,4,6-tri-O-sulfato-1-O-(2′,3′,4′,6′,2,3,6-hepta-O-sulfato-β-D-maltosyl)-D-mannitol; 2,5-Anhydro-3,4-di-O-sulfato-1,6-bis-O-(2,3,4,6-tetra-O-sulfato-α-L-idopyranosyl)-D-mannitol; 2,5-Anhydro-3,4-di-O-sulfato-1,6-bis-O-[2′,3′,4′,6′,2,3,4-hepta-O-sulfato-β-D-maltosyl]-D-mannitol or the sodium or potassium salts or isomers thereof.

7. A pharmaceutical composition comprising a compound as claimed in claim 1, and a pharmaceutically acceptable carrier therefor.

8. Use of compounds according to claim 1 for the preparation of drugs suitable for treating inflammatory disorders of airways of mammals.

9. The use according to claim 8 for the preparation of drugs suitable for treating allergic inflammatory disorders of airways.

10. The use according to claim 9, wherein the allergic inflammatory disorder of the airways is selected from the group consisting of asthma, allergic rhinitis, intrinsic or extrinsic asthma bronchiale, acute or chronic bronchitis, chronic obstructive lung disease, and pulmonary fibrosis.

11. A method for treating an acute or chronic inflammatory disorder of the airways of mammals, comprising administering to a mammal in need of such treatment a therapeutically effective amount of a compound of formula (I).

12. The method of claim 11, wherein the Inflammatory disorder of the airways is an allergic inflammatory disorder.

13. The method of claim 12, wherein the allergic inflammatory disorder of airways is selected from the group consisting of asthma, allergic rhinitis, intrinsic or extrinsic asthma bronchiale, acute or chronic bronchitis, chronic obstructive lung disease, and pulmonary fibrosis.

14. The method of claim 12, wherein the allergic inflammatory disorder of airways is selected from the group consisting of idiopathic pulmonary fibrosis and autoimmune lung disease.

15. The method of claim 11, comprising administering the compound of formula (I) as a single or multiple dose.

16. A process for synthesizing a compound of formula I or pharmaceutically acceptable salt or isomer thereof comprising the step of reacting a compound of formula (VI) 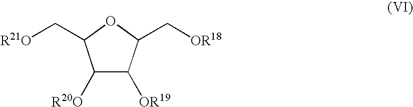
wherein R¹⁸, R¹⁹, R²⁰ and R²¹ are independently selected from H, a glycosyl group or a diglycosyl group or a protected variant thereof with a suitable sulfating reagent to form a compound of formula I or a pharmaceutically acceptable salt or isomer thereof.
